(19) United States
(12) Patent Application Publication
(10) Pub. No.: US 2012/0231468 A1
(43) Pub. Date: Sep. 13, 2012

(54) RNA FROM CYTOLOGY SAMPLES TO DIAGNOSE DISEASE

(75) Inventors: Guy R. Adami, Brookfield, IL (US); Joel L. Schwartz, Chicago, IL (US); Antonia Kolokythas, Skokie, IL (US)

(73) Assignee: BOARD OF TRUSTEES OF THE UNIVERSITY OF ILLINOIS, Urbana, IL (US)

(21) Appl. No.: 13/437,365

(22) Filed: Apr. 2, 2012

Related U.S. Application Data
(63) Continuation-in-part of application No. 12/407,604, filed on Mar. 19, 2009, now Pat. No. 8,153,370.
(60) Provisional application No. 61/037,767, filed on Mar. 19, 2008.

(51) Int. Cl. C12Q 1/68; C12Q 1/02

(52) U.S. Cl. 435/6.12; 435/6.14; 435/29

(57) ABSTRACT

The invention relates to methods and kits for detecting the likelihood that a subject has cancer, e.g., squamous cell carcinoma, by assaying the expression levels of tumor associated genes. More specifically, the expression levels of nucleic acids or proteins can be assayed in the tumor associated genes, e.g., over-expression of beta-2 microgobulin (B2M), keratin 17 (KRT17), interleukin 8 (IL8), or annexin A2 (ANXA2), and under-expression of cytochrome p450 1B1 (CYP1B1) or laminin gamma-2 (LAMC2) can be indicative of the likelihood a subject has squamous cell carcinoma or a precancerous squamous cell disorder. The expression levels compared to standards can be indicative of the likelihood a subject has squamous cell carcinoma. The expression levels of B2M, CYP1B1, KRT17, IL8, ANXA2, or LAMC2 can also be repeatedly assayed to monitor the progression of a squamous cell neoplasia.

### RELATED APPLICATIONS

This application claims the benefit of U.S. Provisional Patent Application Ser. No. 61/037,767, filed Mar. 19, 2008, entitled “RNA From Cytology Samples to Diagnose Disease,” and U.S. patent application Ser. No. 12/407,604, filed Mar. 19, 2009, entitled “RNA From Cytology Samples to Diagnose Disease,” which are both incorporated by reference herein.

### FIELD OF THE INVENTION

The invention relates to methods and kits for detecting the likelihood that a subject has squamous cell carcinoma or neoplasia.

### BACKGROUND OF THE INVENTION

Oral cancer can be any cancerous growth that is found in the mouth. It can arise as a primary lesion originating from any of the oral tissues. The most common form of oral cancer is oral squamous cell carcinoma, originating from the tissues that line the mouth and lips. Most oral cancers are malignant and can spread rapidly. In 2008, in the US alone, more than about 34,000 individuals will be diagnosed with oral cancer. Of these, 66% will be diagnosed with late stage three or four disease.

RNA expression analysis of oral keratinocytes can be used to detect early stages of disease such as oral cancer or to monitor on-going treatment responses of the same or other oral diseases. A limitation is the inability to obtain high quality RNA from oral tissue without using biopsies. While oral cytology cell samples can be obtained from patients in a minimally invasive manner they have not been validated for quantitative analysis of RNA expression.

Obtaining patient RNA without surgery would be an ideal way to facilitate large-scale genetic studies of cancer and simplify patient diagnosis. Because of the accessibility of the oral and cervical mucosa, methods have been in place for some time to examine histologic and genetic variations in normal and tumors cells. Very recently, methods to analyze RNA from cells and fluids from these organs have been explored. Establishing the validity of these approaches for quantification of gene expression remains an important goal.

Analysis of RNA in urine and saliva has the advantage of ease of use for marker discovery, but it has limitations because it does not provide a direct measure of gene expression in the tissue. It measures RNAs that are stable extracellularly, identifying markers that correlate with disease but are less likely to be informative about disease etiology. Potential problems exist. For example, the unknown contribution of RNA from dead and dying cells may not be readily assessed. Also, subtle differences in investigator sampling can accentuate differences in numbers and types of cells isolated.

Accordingly, there exists a need for better methods and kits for detecting the likelihood that a subject has squamous cell carcinoma or neoplasia. Accurate assay techniques for detecting or monitoring such disease states without resort to surgical biopsies would satisfy a long-felt need in the art.

### SUMMARY OF THE INVENTION

Methods and kits are disclosed for detecting the likelihood that a subject has cancer, e.g., squamous cell carcinoma, by assaying the expression levels of tumor associated genes. More specifically, the expression levels of nucleic acids or proteins can be assayed in the tumor associated genes, e.g., beta-2 microgobulin (B2M), cytochrome p450 1B1 (CYP1B1) keratin 17 (KRT17), interleukin 8 (IL8), annexin A2 (ANXA2), or laminin gamma-2 (LAMC2). The expression levels compared to standards can be indicative of the likelihood a subject has squamous cell carcinoma. For example, over-expression of B2M, KRT17, IL8, or ANXA2 and under-expression of LAMC2 or CYP1B1 can be indicative of the likelihood a subject has oral squamous cell carcinoma. Also, over-expression of B2M, KRT17, IL8, or ANXA2, and under-expression of LAMC2 or CYP1B1 can be indicative of the likelihood a subject has a precancerous oral squamous cell disorder.

The expression levels of one or more of B2M, CYP1B1, KRT17, IL8, ANXA2, and LAMC2 can also be repeatedly assayed to monitor the progression of an squamous cell neoplasia.

Methods for assaying gene expression in a tissue sample from a subject by detecting nucleic acid or protein expression level of beta-2 microgobulin (B2M) and at least one additional gene or protein of interest in the sample are disclosed. The over-expression of B2M can be compared to a standard and expression of the additional gene or protein can be compared to a second standard. Differential expression between the assayed genes or proteins of interest and the standards can be indicative of a likelihood that the subject has a presence of or a risk for development of a cancerous squamous cell disorder. Expression levels can be detected simultaneously or separately. The method disclosed herein can include detecting non-degraded nucleic acids that are at least 500 nucleotides in length or partially degraded nucleic acids from dead or dying cells that are less than about 500 nucleotides in length. Moreover, the tissue sample can be from a mouth, lip, tongue, cheek lining, gingiva, palate, skin, esophagus, vagina and cervix of the subject.

In one aspect, a method for detecting the likelihood that a subject has squamous cell carcinoma comprises obtaining a brush cytology sample from a subject, extracting nucleic acids from cells in the sample, and assaying the nucleic acids for expression levels of non-degraded nucleic acid sequences coding for production of B2M, CYP1B1, KRT17, IL8, ANXA2, or LAMC2, wherein over-expression of the B2M, KRT17, IL8, or ANXA2 gene compared to a standard, or under-expression of the LAMC2 or CYP1B1 gene compared to a second standard is indicative of a likelihood that the subject has squamous cell carcinoma.

In another aspect, brush cytology sampling is used to obtain squamous cells suitable for assays. The brush cytology instrument or brush can have one or two cutting or abrasive surfaces. Brushes with one surface can comprise a rod with perpendicular bristles. Brushes with two surfaces can comprise a flat end of the brush and a circular border of the brush. Either surface can be used to obtain the specimen. To obtain the brush cytology sample, firm pressure with a brush can be applied to the area to be sampled. In some embodiments, a brush can be rotated in at least 20 brush strokes, where a single brush stroke is a forward to backward/backward to forward, a side to side or circular movement to obtain the sample. In some other embodiments, a first brush can be rotated in two to five brush strokes, to prime the surface by removing external dead or dying cells and expose underlying layers, then the first brush is discarded. Then a second brush can be rotated in the same location in at least 20 brush strokes to obtain the sample.

In one embodiment, the method further comprises amplifying and quantifying expression of the B2M, CYP1B1, KRT17, IL8, ANXA2, or LAMC2 gene by real time polymerase chain reaction (q-PCR) using primers complementary to an mRNA sequence of at least 15 bases found at least 500 basepairs and preferably at least 1000 basepairs from the encoded 3′ ends of the B2M, CYP1B1, KRT17, IL8, ANXA2, or LAMC2 mRNA transcripts. Amplifying expression products at least 500 basepairs and preferably at least 1000 basepairs from the encoded 3′ ends of the transcripts, corresponding to the transcriptional start site, substantially full length and non-degraded nucleic acid sequences capable of producing proteins of interest, e.g., B2M, CYP1B1, KRT17, IL8, ANXA2, or LAMC2, can be detected. Typically, non-degraded nucleic acid sequences are extracted from living cells taken as part of the sample.

In contrast, degraded nucleic acid sequences are typically extracted from dead cells or cells undergoing apoptosis. Amplifying expression products less than about 500 basepairs or less than about 1000 basepairs in length and corresponding to partially degraded nucleic acid sequences or non-full length gene sequences, like partially degraded nucleic acid sequences from B2M, CYP1B1, KRT17, IL8, ANXA2, or LAMC2, can be detected.

In another aspect, a method is provided for detecting the likelihood that a subject has squamous cell carcinoma, comprising detecting B2M, CYP1B1, KRT17, IL8, ANXA2, or LAMC2 protein or nucleic acid expression levels individually or simultaneously in a sample from the subject, wherein over-expression of the B2M, KRT17, IL8, or ANXA2 gene compared to a standard or under-expression of the LAMC2 or CYP1B1 gene compared to a second standard is indicative of a likelihood that the subject has oral squamous cell carcinoma. Moreover, another aspect is directed to detecting the likelihood that a subject has a precancerous squamous cell disorder, comprising detecting B2M, CYP1B1, KRT17, IL8, ANXA2, or LAMC2 protein or nucleic acid expression levels individually or simultaneously in a sample from the subject, wherein over-expression of the B2M, KRT17, IL8, or ANXA2 gene compared to a standard or under-expression of the LAMC2 or CYP1B1 gene compared to a second standard is indicative of a likelihood that the subject has a precancerous oral squamous cell disorder.

In one aspect, a method for monitoring squamous cell neoplasia in a human subject over time, comprising obtaining a brush cytology sample from a subject at a first time, extracting nucleic acids from cells in the sample, assaying said nucleic acids for the expression level of genes coding for the production of B2M, CYP1B1, KRT17, IL8, ANXA2, or LAMC2, and repeating the steps of obtaining a sample, extracting nucleic acids and assaying for expression levels of B2M, CYP1B1, KRT17, IL8, ANXA2, or LAMC2 at a later time, wherein increased expression of the B2M, KRT17, IL8, or ANXA2 gene at a later time or decreased expression of the LAMC2 or CYP1B1 gene at a later time is indicative of progression of neoplasia. The nucleic acids for the respective genes can be assayed individually or simultaneously, In one embodiment, squamous cell neoplasia in a human subject can be monitored over time in response to a treatment. A sample can be obtained, nucleic acids extracted from the sample, expression level of genes encoding for B2M, CYP1B1, KRT17, IL8, ANXA2, or LAMC2 can be assayed, a treatment can be administered, wherein the treatment is a bioactive agent that leads to an increase in cytochrome p450 proteins, sampling from the subject can be repeated over time and the expression level of B2M, CYP1B1, KRT17, IL8, ANXA2, or LAMC2 at a later time is indicative of the response to the treatment.

In yet another aspect, a kit is provided for assessing the presence of cancer in a sample comprising a pair of primers which specifically hybridize to at least one non-degraded nucleic acid sequence coding for production of B2M, CYP1B1, KRT17, IL8, ANXA2, or LAMC2 gene product and reagents for real-time polymerase chain reaction (q-PCR). In additional embodiments, the kit can comprise additional tools, reagents or instruction manuals. For example, the kit can comprise a brush for obtaining a brush cytology sample from a subject. Also, the kit can comprise a nucleic acid extraction reagent to isolate nucleic acids from a sample.

Further understanding of various aspects of the invention can be obtained by reference to the following detailed description in conjunction with the associated drawings, which are described briefly below.

### BRIEF DESCRIPTION OF THE DRAWINGS

FIG. 1 is a flow chart depicting various steps in an embodiment of a method to detect the likelihood a subject has oral squamous cell carcinoma;

FIG. 2 is flow chart depicting various steps in an embodiment of a method to detect the likelihood a subject has a precancerous oral squamous cell disorder;

FIG. 3 is flow chart depicting various steps in an embodiment of a method to monitor the progression of an oral squamous cell neoplasia over time;

FIG. 4 is flow chart depicting various steps in an exemplary embodiment of a method to detect the likelihood a subject has oral squamous cell carcinoma;

FIG. 5 is flow chart depicting various steps in an exemplary embodiment of a method to detect the likelihood a subject has a precancerous oral squamous cell disorder;

FIG. 6A shows hematoxylin and eosin-stained tissue sections from control unexposed hamster oral tissue (floor of mouth and lateral border of tongue) (bar=200 lm), with one example of stratified squamous epithelium (SSE) labelled;

FIG. 6B shows hematoxylin and eosin-stained tissue sections after 33 weeks of exposure to dibenzo[a,I]pyrene that reveal histopathologic changes characteristic of oral squamous cell carcinoma (bar=200 lm);

FIG. 7A shows a bar graph comparing expression of the B2M gene in brush cytology samples from 13 animals. Each bar represents the relative mRNA level of one of three samples taken on consecutive weeks from oral squamous cell carcinoma tumor in five hamsters and normal mucosa of eight control hamsters. Shown is the mean of three PCR runs of a single sample. For each animal the overall intraclass correlation (ICC) among each set of three measurements was calculated;

FIG. 7B shows a bar graph comparing expression of the CDK2AP1 gene in brush cytology samples from 13 animals;

FIG. 7C shows a bar graph comparing expression of the CYP1B1 gene in brush cytology samples from 13 animals;

FIG. 7D shows a bar graph comparing expression of the GSTP1 gene in brush cytology samples from 13 animals;

FIG. 7E shows a bar graph comparing expression of the PECAM1 gene in brush cytology samples from 13 animals;

FIG. 7F shows a bar graph comparing expression of the VEGF gene in brush cytology samples from 13 animals;

FIG. 8A shows a bar graph comparing measured B2M mRNA levels in brush cytology RNA samples vs. surgically excised (biopsy) tissue from 13 animals. The mean mRNA levels for each tested gene were calculated for brush cytology samples (black bars) vs. surgically removed tissue (white bars)±SEM. The values for the brush cytology cell mRNA were averaged over three separate brush cytology samples. The correlation coefficient (R) comparing the derived values from the two cell sources for each hamster was derived;

FIG. 8B shows a bar graph comparing measured CDK2AP1 mRNA levels in brush cytology RNA samples vs. surgically excised (biopsy) tissue from 13 animals;

FIG. 8C shows a bar graph comparing measured CYP1B1mRNA levels in brush cytology RNA samples vs. surgically excised (biopsy) tissue from 13 animals;

FIG. 8D shows a bar graph comparing measured GSTP1 mRNA levels in brush cytology RNA samples vs. surgically excised (biopsy) tissue from 13 animals;

FIG. 8E shows a bar graph comparing measured PECAM1mRNA levels in brush cytology RNA samples vs. surgically excised (biopsy) tissue from 13 animals;

FIG. 8F shows a bar graph comparing measured VEGF mRNA levels in brush cytology RNA samples vs. surgically excised (biopsy) tissue from 13 animals;

FIG. 9A depicts a brush oral cytology immunofluorescent staining of a mucosal biopsy sample showed cytokeratin staining specifically in the cells of the epithelium (bar=10 lm, BM is basement membrane);

FIG. 9B depicts a brush cytology sample cells were highly enriched for cytokeratin staining;

FIG. 9C shows that brush cytology sample RNA was enriched for epithelial markers CDH1 and CX-26) and depressed for non-epithelial cell markers (DES and VIM) vs. the biopsy sample RNA. RNA was from five control hamsters;

FIG. 10A shows the reproducibility of SPINK5 mRNA gene expression measured from independent cytology samples;

FIG. 10B shows the reproducibility of ECM1 mRNA gene expression measured from independent cytology samples;

FIG. 11A shows the relative expression of B2M in RNA from brush cytology from OSCC and nonmalignant oral lesions and normal controls;

FIG. 11B shows the relative expression of CYP1B1 in RNA from brush cytology from OSCC and nonmalignant oral lesions and normal controls;

FIG. 11C shows the relative expression of KRT17 in RNA from brush cytology from OSCC and nonmalignant oral lesions and normal controls; and

FIG. 12 shows expression of the selected genes measured in RNA from cytology of a subset of 7 oral squamous cell carcinoma (OSCC) samples using QRT-PCR.

### DETAILED DESCRIPTION OF THE INVENTION

RNA analysis from brush oral cytology, on the other hand, has the advantage that live cells can be isolated from a site at risk for a disease such as oral squamous cell carcinoma (OSCC). Early changes in the disease progression that effect gene expression can be detected and because of the minimal invasiveness, the assay can be carried out repeatedly.

Pilot studies from the literature demonstrate that the isolation of RNA from brush oral cytology is possible and that mRNA can be detected using q-PCR or microarray analysis, but it is not clear how reliable the method is and what is being measured. One study indicated that 10-20% of the oral brush cytology mucosal cells from humans were viable as isolated, while we saw somewhat higher numbers from hamsters. In their human study, Spivack et al. saw a qualitative correlation of the detectability of expression of a number of mRNAs in laser microdissected lung tissue and brush cytology cells from the same patients. However, large inter-patient variability in mRNA quantitation was seen (up to 10 000-fold) and the source of this variation was not explored. In another pilot study, RNAs from brush cytology cervical cells were compared to those from a surgically removed cervical tissue specimen by DNA microarray analysis, revealing that similar groups of genes were expressed above background.

Methods are generally provided that relate, in part, to newly discovered correlations between the expression of selected genes, in particular, beta-2 microglobulin (B2M), cytochrome p450 1B1 (CYP1B1), keratin 17 (KRT17), interleukin 8 (IL8), annexin A2 (ANXA2), or laminin gamma-2 (LAMC2) and the presence of cancer, such as, squamous cell carcinoma, in a subject. The relative expression level of the genes, e.g., B2M, CYP1B1, KRT17, IL8, ANXA2, and LAMC2, has been found to be indicative of squamous cell carcinoma in the subject and/or diagnostic of the presence or potential presence of squamous cell carcinoma in a subject. Methods are provided for detecting the likelihood a subject has squamous cell carcinoma, and methods of detecting the likelihood a subject has a precancerous squamous cell disorder by assaying nucleic acids for relative expression levels of B2M, CYP1B1, KRT17, IL8, ANXA2, or LAMC2 genes as compared to a standard.

Also disclosed, at least in part, is the identification of genes which are differentially expressed in samples from squamous carcinoma cells compared to non-cancer cells. A panel of known genes was screened for differential expression patterns in oral brush cytology samples (see Examples 1 and 2). Those genes with statistically significant (p<0.01) differences between the diseased and normal tissues were identified. This differential expression was observed either as a decrease in expression, or an increase in expression.

Accordingly, methods are provided for the analysis of B2M, CYP1B1, KRT17, IL8, ANXA2, and LAMC2 genes, the corresponding mRNA transcripts, and the encoded polypeptides, as an indication for the presence of or risk for development of, and the progression of squamous cell carcinoma. Overexpression of the B2M, KRT17, IL8, or ANXA2 gene can be indicative of the presence of disease and a precancerous oral squamous cell disorder. Underexpression of the LAMC2 or CYP1B1 gene can also be indicative of the likelihood a subject has a precancerous oral squamous cell disorder, while the underexpression of the LAMC2 or CYP1B1 gene can be indicative the subject has oral squamous cell carcinoma.

Detection of the presence or expression levels of non-degraded or partially degraded nucleic acid sequences, e.g., non-degraded sequences can be at least 500 basepairs and preferably at least 1000 basepairs from the encoded 3′ ends of the B2M, CYP1B1, KRT17, IL8, ANXA2, or LAMC2 mRNA transcripts and partially degraded sequences can be less than about 500 basepairs or less than about 1000 basepairs in length, can be performed using methods known in the art.

Typically, it can be convenient to assess the presence and/or quantity of mRNA or cDNA by real-time polymerase chain reaction (q-PCR) or quantitative-PCR (q-PCR), in which mRNA can be isolated from a cell or tissue sample, converted to cDNA using reverse transcriptase by methods known in the art, hybridized with gene specific oligonucleotides (e.g., B2M, CYP1B1, KRT17, IL8, ANXA2, or LAMC2 primers), and amplified in the presence of probe or diagnostic label. The label group can be a fluorescent compound. Other useful methods of mRNA detection and/or quantification include northern blot, gel electrophoresis, column chromatography, q-PCR, and other methods known by one skilled in the art.

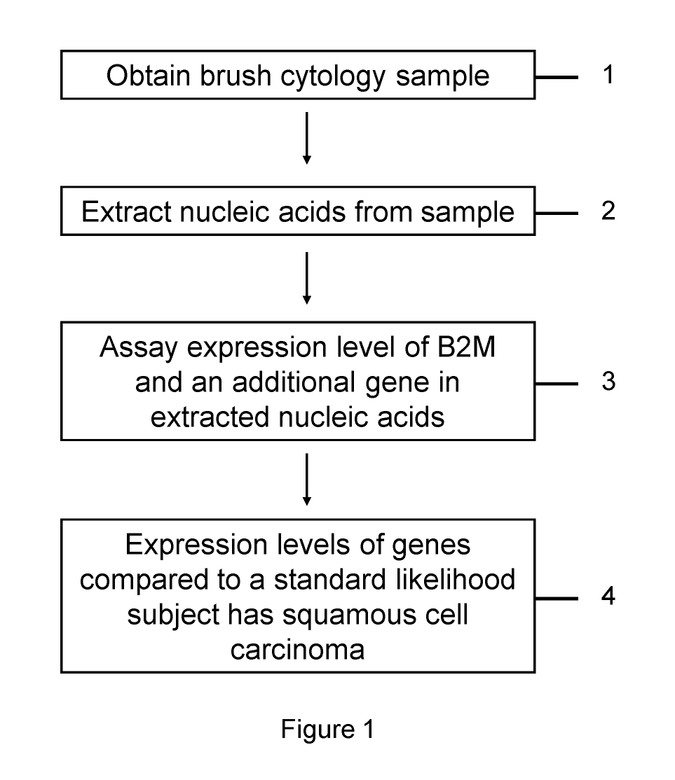

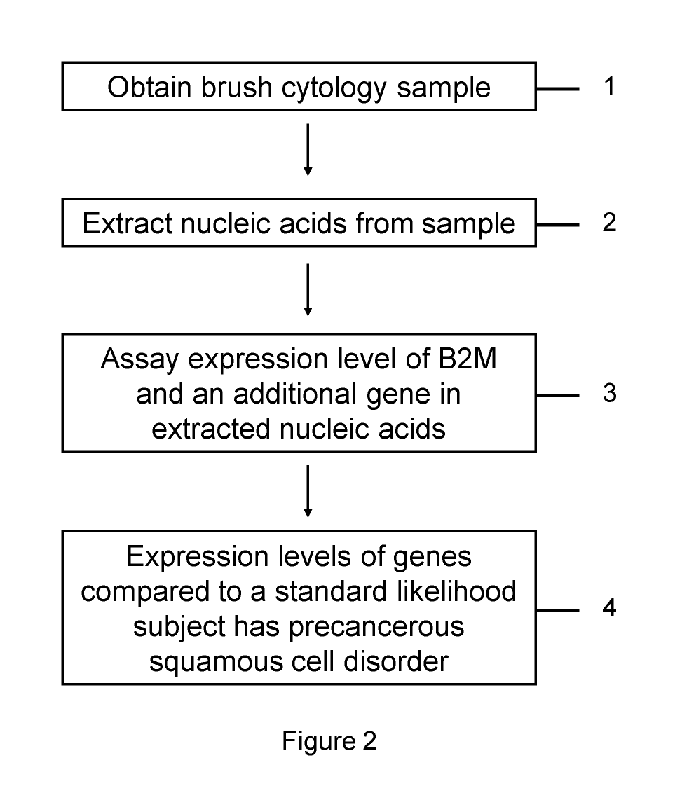

In another aspect, a method is provided for detecting the likelihood that a subject has squamous carcinoma by assaying expression level of B2M, CYP1B1, KRT17, IL8, ANXA2, or LAMC2 genes, whose quantity or expression level is assayed for the likelihood that a subject has squamous carcinoma (FIG. 1). The genes, e.g., B2M, CYP1B1, KRT17, IL8, ANXA2, and LAMC2, are either increased or decreased in expression level in the cancer tissue in a fashion that is either positively or negatively indicative of the subject having squamous cell carcinoma. In yet another aspect, a method is provided for detecting the likelihood that a subject has a precancerous squamous cell disorder by assaying the expression levels of B2M, CYP1B1, KRT17, IL8, ANXA2, or LAMC2 genes (FIG. 2). The genes are either increased or decreased in expression level that can be indicative that the subject has a precancerous squamous cell disorder.

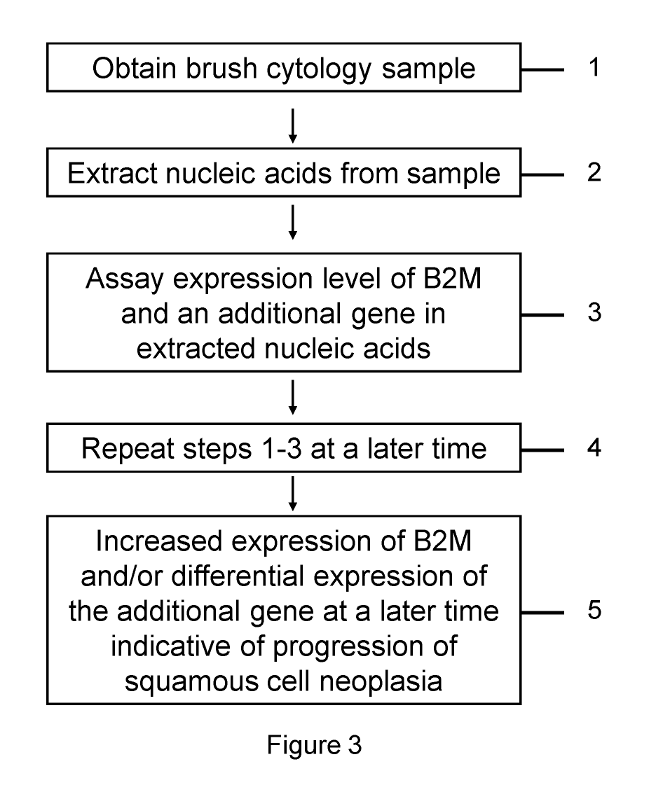

In yet another aspect, a method is provided for monitoring squamous cell neoplasia in a human subject over time by assaying the expression level of B2M, CYP1B1, KRT17, IL8, ANXA2, or LAMC2 genes, whose expression level is assayed for the likelihood that a subject has squamous carcinoma (FIG. 3).

The terms used in this invention adhere to the standard definitions generally accepted by those having ordinary skill in the art. In case any further explanation might be needed, some terms have been elucidated below and throughout the application.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the terms “polynucleotide,” “oligonucleotide” and “nucleic acid sequences” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, complementary DNA (cDNA), recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also includes both double- and single-stranded molecules. Unless otherwise specified or required, any embodiment that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. This, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein can be used to identify larger fragments or full-length coding sequences of the gene with which they are associated. Methods of isolating larger fragment sequences are known to those of skill in the art, some of which are described herein. Previously known and uncharacterized polymorphisms in B2M, CYP1B1, KRT17, IL8, ANXA2, or LAMC2 genes are also included. In addition, alternative splicing products that produce variation in the mRNA expression pattern are also included.

A “gene product” includes an amino acid (e.g., peptide or polypeptide) generated when a gene is transcribed and translated.

The terms “tumor-associated genes” as used herein refers to a gene(s) found to be differentially expressed, either over-expressed or under-expressed in cancer tissue and originally identified by their differential expression in cancer cells compared to non-cancer cells.

The term “non-degraded” nucleic acid sequences as used herein refers to substantially full length nucleic acid sequences capable of producing proteins of interest or at least more than 500 nucleotides in length and corresponding to the protein of interest, e.g., B2M, CYP1B1, KRT17, IL8, ANXA2, or LAMC2. Typically, non-degraded nucleic acid sequences are extracted from living cells taken as part of the sample. Amplifying expression products at least 500 basepairs and preferably at least 1000 basepairs from the encoded 3′ ends of the mRNA transcripts, corresponding to the transcriptional start site, substantially full length and non-degraded nucleic acid sequences capable of producing proteins, e.g., B2M, CYP1B1, KRT17, IL8, ANXA2, or LAMC2, can be detected. Alternatively, non-degraded nucleic acid sequences preferable for the assay purposes disclosed herein are typically at least 50 percent or more of the full-length gene and suitable primers can be used to selectively amplify such nucleic acid sequences.

The terms “partially degraded” nucleic acid sequences as used herein refers to non-full length nucleic acid sequences whose full length sequences are capable of producing proteins of interest, e.g., B2M, CYP1B1, KRT17, IL8, ANXA2, or LAMC2. Typically, partially degraded nucleic acid sequences are extracted from dead or dying cells or cells undergoing apoptosis taken as part of the sample. Amplifying expression products less than about 500 basepairs or less than about 1000 basepairs in length whose full length sequences correspond to the protein of interest, e.g., B2M, CYP1B1, KRT17, IL8, ANXA2, or LAMC2. Alternatively, partially degraded nucleic acid sequences for the assays disclosed herein are typically at least 50 percent or less of the full-length gene and suitable primers can be used to selectively amplify such nucleic acid sequences.

A “probe” when used in the context of polynucleotide manipulation includes a reagent to detect a target present in a sample of interest by hybridizing or incorporation with the target. Usually, a probe will comprise a label or a means by which a label can be attached or incorporated with the target. Suitable labels include, but are not limited to fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are well known in the art, and are taught, for example, in MacPherson et al., IRL Press at Oxford University Press (1991)). “Quantitative PCR” (“q-PCR”), also referred herein as real-time PCR (q-PCR), is based on PCR to amplify and simultaneously quantify a target DNA molecule. All processes of producing replicate copies of a polynucleotide, such as PCR or gene cloning, are collectively referred to herein as “replication.” A primer can also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism generated into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence can usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences. Various splice acceptor sites can be necessary for RNA splicing and can be included herein within the definition of “control sequences.” Some such sequences also play a role in the abundance and stage-specificity of gene expression.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression can include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector can include a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Differentially expressed”, as applied to a gene, includes the differential production of mRNA transcribed from a gene or a protein product encoded by the gene. A differentially expressed gene may be overexpressed or underexpressed as compared to the expression level of a normal, control cell or standard. In one aspect, it includes a differential that can be 1.5 times, preferably 2 times or preferably greater than 2 times higher or lower than the expression level detected in a control sample. The term “differentially expressed” can also include nucleotide sequences in a cell or tissue which are expressed where silent in a control cell or not expressed where expressed in a control cell.

The term “polypeptide” includes a compound of two or more subunit amino acids. The subunits can be linked by peptide bonds. In another embodiment, the subunit can be linked by other bonds, e.g., ester, ether, etc. As used herein the term “amino acid” includes either natural and/or unnatural or synthetic amino acids, including glycine and both the D or L optical isomers. A peptide of three or more amino acids can commonly be referred to as an oligopeptide. Peptide chains of greater than three or more amino acids can be referred to as a polypeptide or a protein.

“Hybridization” includes a reaction in which one or more polynucleotides react to form a complex that can be stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PCR reaction, or the enzymatic cleavage of a polynucleotide by a ribozyme.

A nucleic acid molecule can be “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989, supra). The conditions of temperature and ionic strength determine the “stringency” of the hybridization. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. Preferably a minimum length for a hybridizable nucleic acid can be at least about 10 nucleotides; more preferably at least about 15 nucleotides.

Hybridization reactions can be performed under conditions of different “stringency”. The stringency of a hybridization reaction includes the difficulty with which any two nucleic acid molecules can hybridize to one another. Under stringent conditions, nucleic acid molecules at least 60%, 65%, 70%, 75%, 80%, 85%, 90%, 95%, 97%, 98%, 99% identical to each other remain hybridized to each other, whereas molecules with low percent identity cannot remain hybridized.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprises an antigen recognition site of the required specificity.

Proteins or polypeptides can be detected by contacting the protein with an antibody-based binding moiety that specifically binds to the protein of interest, or to a fragment of that protein. Formation of the antibody-protein complex is then detected and measured to indicate protein levels. Anti-protein antibodies can be obtained commercially (e.g. human protein affinity purified polyclonal and monoclonal antibodies). Alternatively, antibodies can be raised against the protein of interest, or a portion of that protein. Antibodies can also be produced using standard methods to produce antibodies, for example, by monoclonal antibody production.

Antibody based binding moieties for the detection of a protein, the level of the protein of interest can correlate to the intensity of the signal emitted from the detectably labeled antibody. Antibody-based binding moieties can be detectably labeled by linking the antibody to an enzyme. For example, chemiluminescence is a method that can be used to detect an antibody-based binding moiety. Detection may also be accomplished using any of a variety of other immunoassays. For example, by radioactively labeling an antibody can detect the antibody through the use of radioimmune assays. It is also possible to label an antibody with a fluorescent compound. Among the most commonly used fluorescent labeling compounds are CYE dyes, fluorescein isothiocyanate, rhodamine, phycoerytherin, phycocyanin, allophycocyanin, o-phthaldehyde and fluorescamine. An antibody can also be detectably labeled using fluorescence emitting metals such as ¹⁵²Eu, or similar other metals.

Protein levels can be measured by immunoassays, such as enzyme linked immunoabsorbant assay (ELISA), radioimmunoassay (RIA), immunoradiometric assay (IRMA), western blotting, or immunohistochemistry. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety. ELISAs are widely used enzyme immunoassays. There are different forms of ELISA, such as “sandwich ELISA” and “competitive ELISA” which are well known in the art.

The term “cancerous” as used herein is intended to refer to any abnormal cells that divide without control characterized by the proliferation of anaplastic cells that can invade surrounding tissues and metastasize to new body sites.

The term “oral cancer” as used herein refers to any cancerous tissue growth located in the mouth. It can arise as a primary lesion originating in any of the oral tissues, by metastasis from a distant site of origin, or by extension from a neighboring anatomic structure. Oral cancers can originate in any of the tissues of the mouth. The most common oral cancer is squamous cell carcinoma, originating in the tissues that line the mouth and lips. Oral or mouth cancer most commonly involves the tissue of the lips or the tongue. Oral cancer can also occur on the floor of the mouth, cheek lining, gingiva (gums), or the palate (roof of the mouth). Many oral cancers can be malignant and can spread rapidly. Oral cells can include, but are not limited to, pseudostratified epithelium, columnar epithelium and a variety of squamous epithelium: keratinized, non-keratinized and stratified.

The terms “squamous cell carcinoma” refer to a type of cancer that can occur in a variety of organs, including, but not limited to: lips, skin, mouth, esophagus, vagina and cervix.

The term “subject” refers to any living organism. The term subject comprises, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. In preferred embodiments, the subject is a mammal, including humans and non-human mammals. In a more preferred embodiment, the subject is a mammal. In the most preferred embodiment, the subject is a human.

The terms “sample,” “sample from a subject” and “extracted sample” as used herein refer to a small quantity of tissue from a subject, which can be obtained, e.g., by employing methods known in the art. Such a tissue sample, e.g., brush cytology sample, can contain cancer cells, non-cancer cells or both. The term sample comprises, but is not limited to, oral tissues, oral cells from the mouth, lips, tongue, cheek lining, gingiva, palate, skin, esophagus, vagina and cervix of a subject.

The term “standard” as used herein refers to a control sample. The “standard” expression levels can be detected, for example, in non-cancer samples, normal subjects without cancer or untreated samples. The “standard” expression level can also refer to nucleic acid expression levels or protein levels present in non-cancer samples, normal subjects without cancer or untreated samples. Standards can provide a control or comparison for determining the outcome of the experiment. Internal “standard” refers to an experimental optimal control to determine the consistency of an experiment or set of experiments. An example of internal standards can be potential housekeeping genes identified on their constant expression in many tissues or on consistent levels in normal and tumor tissue.

Various aspects of the invention are described in further detail in the following subsections:

### I. Beta-2 Microglobulin (B2M)

Beta-2 microglobulin (B2M) (NM_{—}004048) (SEQ ID.: 1) is a component of the major histocompatibility complex (MHC) class I molecules, which are present on almost all nucleated cells of the body. B2M lies lateral to the alpha3 chain on the cell surface and lacks a transmembrane domain. It interacts with the alpha chains and class I-like molecules, which are important for antigen presentation.

Beta-2-microglobulin has been found in the serum of normal individuals and in the urine in elevated amounts in patients with Wilson disease, cadmium poisoning, and other conditions leading to renal tubular dysfunction.

Previous studies have found that some tumors lack cell surface expression of HLA class I molecules and this can be one mechanism by which tumor cells escape immune recognition by cytotoxic T cells. In some cases, tumor escape is due to loss of the heavy chain surface expression encoded by the HLA-A, -B, and -C genes; in other cases, defects in expression of the B2M gene for the light chain can be responsible.

The Daudi lymphoblastoid cell line, derived from a patient with Burkitt lymphoma and lacking both HLA antigens and beta-2 microglobulin, fails to express HLA class I molecules because of a specific defect in the B2M component. In the human melanoma cell line FO-1, it was found that the lack of expression of HLA class I antigens was the result of a defect in the B2M gene: a deletion of the first exon of the 5-prime flanking region and of a segment of the first intron. Analyses using single-strand conformation polymorphism (SSCP) analysis to screen a series of 37 established colorectal cell lines, 22 fresh tumor samples, and 22 normal DNA samples for mutations in the B2M gene, found mutations in 6 of 7 colorectal cell lines and 1 of 22 fresh tumors, whereas no mutations were detected in the normal DNA samples. Sequencing of these mutations showed that an 8-bp CT repeat in the leader peptide sequence was particularly variable, since 3 of the cell lines and 1 fresh tumor sample had deletions in this region. In 2 related colorectal cell lines, DLD-1 and HCT-15, 2 similar mutations were identified. Expression of beta-2-microglobulin was examined using a series of monoclonal antibodies in an ELISA system and reduced expression was correlated with a mutation in 1 allele of the B2M gene, whereas loss of expression was seen in instances where a line was homozygous for a mutation or heterozygous for 2 mutations.

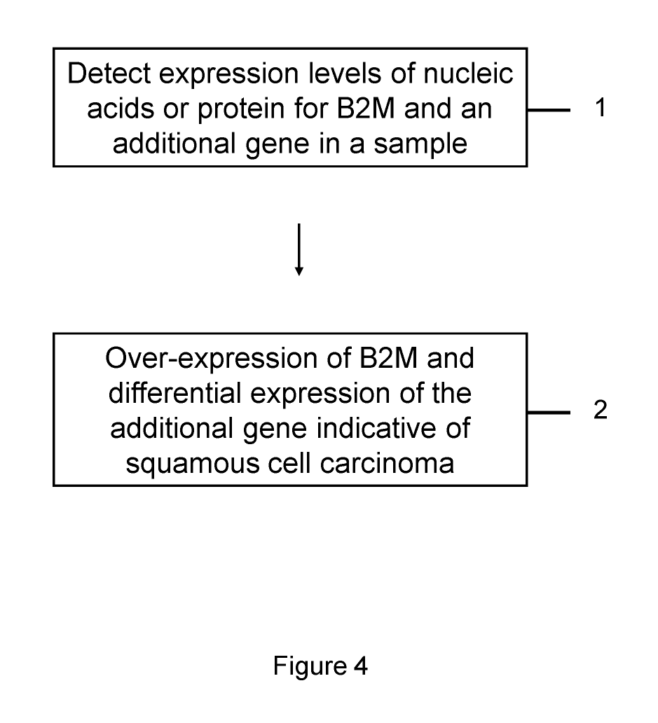

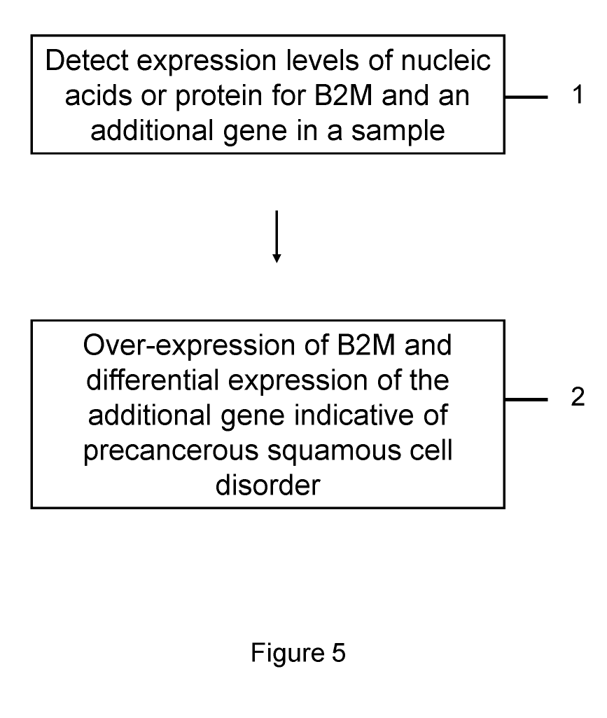

The methods disclosed provide, in part, a method to detect expression level changes in tumor-associated genes, such as changes in B2M gene expression levels, in brush cytology samples. In one aspect, the nucleic acid expression level, e.g., increased expression, of the B2M gene is indicative of a likelihood that a subject has squamous cell carcinoma (FIG. 1). In another aspect, protein or nucleic acid expression level, e.g., increased expression, of B2M is compared to a standard and is indicative of a likelihood that the subject has squamous cell carcinoma (FIG. 4). In yet another aspect, protein or nucleic acid expression levels, e.g., increased expression, of the B2M gene compared to a standard is indicative of a likelihood that the subject has a precancerous squamous cell disorder (FIG. 5). In one aspect, nucleic acids expression level of B2M is assayed over time, at repeated intervals, and expression level, e.g., increased expression, of the B2M gene is indicative of progression of neoplasia (FIG. 3). In another aspect, nucleic acids expression level of B2M is compared to a standard and over-expression of B2M is indicative of a likelihood that the subject has a precancerous squamous cell disorder (FIG. 2). In yet another aspect, a kit is provided for assessing the presence of cancer in a sample comprising a pair of primers that specifically hybridize to at least one non-degraded B2M nucleic acid sequences and reagents for real-time PCR.

### II. Cytochrome P450 Proteins

Cytochrome p450 proteins are a large and diverse family of hemoproteins and monooxygenases which catalyze reactions involved in drug metabolism and synthesis of cholesterol, steroids and other lipids and responsible for the phase 1 metabolism of a wide range of structurally diverse substrates by inserting 1 atom of atmospheric oxygen into the substrate molecule, thereby creating a new functional group (e.g., —OH, —NH2, —COOH). Some well known family members include: cytochrome p450 and cytochrome p450 1A1 (CYP1A1). While less studied and more recently discovered, cytochrome p450 1B1 (CYP1B1) (NM_{—}000104) (SEQ ID.: 2) also belongs to the cytochrome 450 superfamily of proteins. CYP1B1 was originally identified in 1994 through its homology to other identified family members, such as CYP1A1 with 44% identity. Despite the similarity, the two enzymes have very different catalytic efficiencies and metabolites when incubated with common substrates. CYP1B1 has also been found to be regulated by the aryl hydrocarbon receptor, a ligand activated transcription factor, and is expressed in many normal human tissues.

Recently CYP1B1 has been shown to be important in fetal development, with mutations linked to a form of primary congenital glaucoma. Screening for the presence of coding sequence changes in the CYP1B1 gene identified 3 different truncating mutations: a 13-bp deletion found in 1 consanguineous and 1 nonconsanguineous family; a single cytosine insertion observed in another 2 consanguineous families; and a large deletion found in an additional consanguineous family. In addition, a G-to-C transversion at nucleotide 1640 of the CYP1B1 coding sequence was found that caused a val432-to-leu amino acid substitution. This change created an EcoR57 restriction site, thus providing a rapid screening method. Heterozygosity for the val432-to-leu change was found in 51.4% of 70 normal individuals. This amino acid change was not in that part of CYP1B1 that represented conserved sequences, and both valine and leucine are neutral and hydrophobic. Their very similar aliphatic side groups differ by a single —CH2 group. Therefore, this change appeared to represent a common amino acid polymorphism that is not related to the primary congenital glaucoma phenotype. However the finding was not unexpected, as a link between members of this superfamily and the processes of growth and differentiation had been postulated previously. They speculated that CYP1B1 participates in the metabolism of an as-yet-unknown biologically active molecule that is a participant in eye development.

Methods generally provided include a method for detecting tumor-associated changes in the expression level in genes, such as changes in CYP1B1 expression levels, in brush cytology samples. In one aspect, the nucleic acid expression level, e.g., decreased expression, of the CYP1B1 gene is indicative of a likelihood that a subject has squamous cell carcinoma (FIG. 1). In another aspect, protein or nucleic acid expression level, e.g., decreased expression, of CYP1B1 is compared to a standard and is indicative of a likelihood that the subject has squamous cell carcinoma (FIG. 4). In yet another aspect, protein or nucleic acid expression levels, e.g., decreased expression, of the CYP1B1 gene compared to a standard is indicative of a likelihood that the subject has a precancerous squamous cell disorder (FIG. 5). In one aspect, nucleic acid expression level of CYP1B1 can be monitored over time, at repeated intervals, and the expression level, e.g., decreased expression, of the CYP1B1 gene is indicative of progression of squamous cell neoplasia (FIG. 3). In another aspect, nucleic acids expression level of CYP1B1 is compared to a standard and under-expression of CYP1B1 is indicative of a likelihood that the subject has a precancerous squamous cell disorder (FIG. 2). In yet another aspect, a kit is provided for assessing the presence of cancer in a sample comprising a pair of primers that specifically hybridize to at least one non-degraded CYP1B1 nucleic acid sequences and reagents for real-time RT-PCR.

Another aspect relates to increasing expression of CYP1B1, and other cytochrome p450 family members, such as CYP1A1, as a method to inhibit carcinogenesis. Bioactive agents have been characterized to increase cytochrome p450-metabolism, and related family members-metabolism, of certain medications leading to increased bioavailability. In another aspect, administering activators of cytochrome p450 can be useful in treating or inhibiting squamous cell neoplasia. The activators can be a bioactive agent that leads to increased cytochrome p450 proteins and at least one of cytochrome p450 1B1 (CYP1B1), cytochrome p450 1A1 (CYP1A1) and combinations thereof.

### III. Keratin 17 (KRT17)

Keratin 17 (KRT17)(BC000159) (SEQ ID.: 3), also known as type 1 cytoskeletal 17, encodes for a protein in humans that is found in the nail beds, hair follicles, sebaceous glands, and other epidermal appendages.

Required for the correct growth of hair follicles, in particular for the persistence of the anagen (growth) state. Modulates the function of TNF-alpha in the specific context of hair cycling. Regulates protein synthesis and epithelial cell growth through binding to the adapter protein SFN and by stimulating Akt/mTOR pathway. Involved in tissue repair.

Expression of KRT17 is primarily in the outer root sheath and medulla region of hair follicle specifically from eyebrow and beard, digital pulp, nail matrix and nail bed epithelium, mucosal stratified squamous epithelia and in basal cells of oral epithelium, palmoplantar epidermis and sweat and mammary glands. It has also been shown to be expressed in myoepithelium of prostate, basal layer of urinary bladder, cambial cells of sebaceous gland and in exocervix.

KRT17 may play a role in the formation and maintenance of various skin appendages, specifically in determining shape and orientation of hair. It has also been implicated as a marker of basal cell differentiation in complex epithelia and indicative of a certain type of epithelial stem cell. In addition, KRT17 may act as an autoantigen in the immunopathogenesis of psoriasis, with certain peptide regions being a major target for autoreactive T-cells and hence causing their proliferation.

Defects in KRT17 are known to cause of pachyonychia congenita type 2 (PC2), also known as pachyonychia congenita Jackson-Lawler type. PC2 is an autosomal dominant ectodermal dysplasia characterized by hypertrophic nail dystrophy resulting in onchyogryposis (thickening and increase in curvature of the nail), palmoplantar keratoderma and hyperhidrosis, follicular hyperkeratosis, multiple epidermal cysts, absent/sparse eyebrow and body hair, and by the presence of natal teeth. Defects in KRT17 are also a cause of steatocystoma multiplex (SM). SM is a disease characterized by round or oval cystic tumors widely distributed on the back, anterior trunk, arms, scrotum, and thighs.

Methods provided include detecting tumor-associated changes in the expression level of genes, such as changes in KRT17 expression levels, in brush cytology samples. In one aspect, the protein or nucleic acid expression level, e.g., increased expression, of the KRT17 gene is indicative of a likelihood that a subject has squamous cell carcinoma. In another aspect, protein or nucleic acid expression level, e.g., increased expression, of KRT17 is compared to a standard and is indicative of a likelihood that the subject has squamous cell carcinoma. In yet another aspect, protein or nucleic acid expression levels, e.g., increased expression, of the KRT17 gene compared to a standard is indicative of a likelihood that the subject has a precancerous squamous cell disorder. In one aspect, nucleic acid expression level of KRT17 can be monitored over time, at repeated intervals, and the expression level, e.g., increased expression, of the KRT17 gene is indicative of progression of squamous cell neoplasia. In another aspect, nucleic acids expression level of KRT17 is compared to a standard and over-expression of KRT17 is indicative of a likelihood that the subject has a precancerous squamous cell disorder. In yet another aspect, a kit is provided for assessing the presence of cancer in a sample comprising a pair of primers that specifically hybridize to at least one non-degraded KRT17 nucleic acid sequences and reagents for real-time RT-PCR.

### IV. Interleukin 8 (IL8)

Interleukin 8 (IL8)(BC013615) (SEQ ID.: 4) encodes for a member of the CXC chemokine family. Surface membrane receptors capable of binding to IL-8 include the most frequently studied G-protein-coupled receptors, CXCR1 and CXCR2. Expression and affinity to IL-8 is different in the two receptors.

This chemokine is one of the major mediators of the inflammatory response and is secreted by several cell types, such as macrophages and epithelial cells. It is also synthesized by endothelial cells, which store IL8 in vesicles. IL-8 can be secreted by any cell with toll-like receptors and are involved in the innate immune response.

IL-8's primary function is to recruit neutrophils to phagocytose antigens, which trigger the antigen pattern toll-like receptors. It functions as a chemoattractant, and is also a potent angiogenic factor. Both the monomer and the homodimer forms of IL-8 have been reported as potent inducers of CXCR1 and CXCR2. The homodimer is more potent. However, methylation of Leu25 can block activity of the dimers.

Interleukin-8 is often associated with inflammation. As an example, it has been cited as a proinflammatory mediator in gingivitis and psoriasis. The fact that Interleukin-8 secretion is increased by oxidant stress, which thereby causes the recruitment of inflammatory cells induces a further increase in oxidant stress mediators, making it a key parameter in localized inflammation. IL-8 is also believed to play a role in the pathogenesis of bronchitis, a common respiratory tract disease caused by viral infection.

Provided are methods for generally detecting tumor-associated changes in the expression level in genes, such as changes in IL8 expression levels, in brush cytology samples. In one aspect, the protein or nucleic acid expression level, e.g., increased expression, of the IL8 gene is indicative of a likelihood that a subject has squamous cell carcinoma. In another aspect, protein or nucleic acid expression level, e.g., increased expression, of IL8 is compared to a standard and is indicative of a likelihood that the subject has squamous cell carcinoma. In yet another aspect, protein or nucleic acid expression levels, e.g., increased expression, of the IL8 gene compared to a standard is indicative of a likelihood that the subject has a precancerous squamous cell disorder. In one aspect, nucleic acid expression level of IL8 can be monitored over time, at repeated intervals, and the expression level, e.g., increased expression, of the IL8 gene is indicative of progression of squamous cell neoplasia. In another aspect, nucleic acids expression level of IL8 is compared to a standard and over-expression of IL8 is indicative of a likelihood that the subject has a precancerous squamous cell disorder. In yet another aspect, a kit is provided for assessing the presence of cancer in a sample comprising a pair of primers that specifically hybridize to at least one non-degraded IL8 nucleic acid sequences and reagents for real-time RT-PCR.

### V. Annexin A1 and Annexin A2 (ANXA1 and ANXA2)

Annexin A1 (ANXA1)(BC035993) (SEQ ID.: 5) encodes a protein also known as lipocortin 1. Annexin I belongs to the annexin family of Ca²⁺-dependent phospholipid-binding proteins that have a molecular weight of approximately 35,000 to 40,000 and are preferentially located on the cytosolic face of the plasma membrane. Annexin I protein has an apparent relative molecular mass of 40 kDa, with phospholipase A2 inhibitory activity.

Annexin AI has been of interest for use as a potential anticancer drug. Upon induction by modified nonsteroidal anti-inflammatory drugs and other potent anti-inflammatory drugs, annexin I inhibits the NF-kappaB signal transduction pathway, which is exploited by cancerous cells to proliferate and avoid apoptosis. ANXA1 inhibits the activation of NF-κB by binding to the p65 subunit.

Generally provided is a method for detecting tumor-associated changes in the expression level in genes, such as changes in ANXA1 expression levels, in brush cytology samples. In one aspect, the protein or nucleic acid expression level, e.g., increased expression, of the ANXA1 gene is indicative of a likelihood that a subject has squamous cell carcinoma. In another aspect, protein or nucleic acid expression level, e.g., increased expression, of ANXA1 is compared to a standard and is indicative of a likelihood that the subject has squamous cell carcinoma. In yet another aspect, protein or nucleic acid expression levels, e.g., increased expression, of the ANXA1 gene compared to a standard is indicative of a likelihood that the subject has a precancerous squamous cell disorder. In one aspect, nucleic acid expression level of ANXA1 can be monitored over time, at repeated intervals, and the expression level, e.g., increased expression, of the ANXA1 gene is indicative of progression of squamous cell neoplasia. In another aspect, nucleic acids expression level of ANXA1 is compared to a standard and over-expression of ANXA1 is indicative of a likelihood that the subject has a precancerous squamous cell disorder. In yet another aspect, a kit is provided for assessing the presence of cancer in a sample comprising a pair of primers that specifically hybridize to at least one non-degraded ANXA1 nucleic acid sequences and reagents for real-time RT-PCR.

Annexin A2 (ANXA2)(BC093056) (SEQ ID.: 6) gene has three pseudogenes located on chromosomes 4, 9 and 10, respectively. Multiple alternatively spliced transcript variants encoding different isoforms have been found for this gene. The gene encodes a protein also known as annexin A2. Annexin A2 is involved in diverse cellular processes such as cell motility, linkage of membrane-associated protein complexes to the actin cytoskeleton, endocytosis, fibrinolysis, ion channel formation, and cell matrix interactions. It is a calcium-dependent phospholipid-binding protein whose function is to help organize exocytosis of intracellular proteins to the extracellular domain. Annexin A2 is a pleiotropic protein meaning that its function is dependent on place and time in the body.

Generally provided is a method for detecting tumor-associated changes in the expression level in genes, such as changes in ANXA2 expression levels, in brush cytology samples. In one aspect, the protein or nucleic acid expression level, e.g., increased expression, of the ANXA2 gene is indicative of a likelihood that a subject has squamous cell carcinoma. In another aspect, protein or nucleic acid expression level, e.g., increased expression, of ANXA2 is compared to a standard and is indicative of a likelihood that the subject has squamous cell carcinoma. In yet another aspect, protein or nucleic acid expression levels, e.g., increased expression, of the ANXA2 gene compared to a standard is indicative of a likelihood that the subject has a precancerous squamous cell disorder. In one aspect, nucleic acid expression level of ANXA2 can be monitored over time, at repeated intervals, and the expression level, e.g., increased expression, of the ANXA2 gene is indicative of progression of squamous cell neoplasia. In another aspect, nucleic acids expression level of ANXA2 is compared to a standard and over-expression of ANXA2 is indicative of a likelihood that the subject has a precancerous squamous cell disorder. In yet another aspect, a kit is provided for assessing the presence of cancer in a sample comprising a pair of primers that specifically hybridize to at least one non-degraded ANXA2 nucleic acid sequences and reagents for real-time RT-PCR.

### VI. Laminin Gamma-2 (LAMC2 or LMNB)

Laminins are a family of extracellular matrix glycoproteins and make up the major noncollagenous constituent of basement membranes. They have been implicated in a wide variety of biological processes including cell adhesion, differentiation, migration, signaling, neurite outgrowth and metastasis.

Laminins are composed of 3 non identical chains: laminin alpha, beta and gamma, formerly A, B1, and B2, respectively. Laminins form a cruciform structure consisting of 3 short arms, each formed by a different chain, and a long arm composed of all 3 chains. Each laminin chain is a multidomain protein encoded by a distinct gene.

(Laminin gamma-2 (LAMC2)(BC113378) (SEQ ID.: 7) encodes the gamma-2 chain isoform. The gamma-2 chain, formerly thought to be a truncated version of beta chain (B2t), is highly homologous to the gamma-1 chain. However, gamma-2 lacks domain VI, and domains V, IV and III are shorter than gamm-1. It is differentially expressed in several fetal tissues from gamma-1, and is specifically localized to epithelial cells in skin, lung and kidney.

Laminin gamma-2 together with alpha-3 and beta-3 chains constitute laminin 5 (earlier known as kalinin), which is an integral part of the anchoring filaments that connect epithelial cells to the underlying basement membrane. The epithelium-specific expression of laminin gamma-2 implied a role as an epithelium attachment molecule, and mutations in this gene have been associated with the skin disease, junctional epidermolysis bullosa.

Disclosed are methods for detecting tumor-associated changes in the expression level in genes, such as changes in LAMC2 expression levels, in brush cytology samples. In one aspect, the protein or nucleic acid expression level, e.g., decreased expression, of the LAMC2 gene is indicative of a likelihood that a subject has squamous cell carcinoma. In another aspect, protein or nucleic acid expression level, e.g., decreased expression, of LAMC2 is compared to a standard and is indicative of a likelihood that the subject has squamous cell carcinoma. In yet another aspect, protein or nucleic acid expression levels, e.g., decreased expression, of the LAMC2 gene compared to a standard is indicative of a likelihood that the subject has a precancerous squamous cell disorder. In one aspect, nucleic acid expression level of LAMC2 can be monitored over time, at repeated intervals, and the expression level, e.g., decreased expression, of the LAMC2 gene is indicative of progression of squamous cell neoplasia. In another aspect, nucleic acids expression level of LAMC2 is compared to a standard and under-expression of LAMC2 is indicative of a likelihood that the subject has a precancerous squamous cell disorder. In yet another aspect, a kit is provided for assessing the presence of cancer in a sample comprising a pair of primers that specifically hybridize to at least one non-degraded LAMC2 nucleic acid sequences and reagents for real-time RT-PCR.

### VII. Extracellular Matrix Protein 1 (ECM1)

Extracellular matrix protein 1 (ECM1)(BC023505) (SEQ ID.: 8) encodes an extracellular protein containing motifs with a cysteine pattern characteristic of the cysteine pattern of the ligand-binding “double-loop” domains of the albumin protein family. This gene maps outside of the epidermal differentiation complex (EDC), a cluster of three gene families involved in epidermal differentiation. Alternatively spliced transcript variants encoding distinct isoforms have also been described.

The encoded protein is a soluble protein involved in endochondral bone formation, angiogenesis, and tumor biology. It also interacts with a variety of extracellular and structural proteins, contributing to the maintenance of skin integrity and homeostasis. Mutations in this gene are associated with lipoid proteinosis disorder (also known as hyalinosis cutis et mucosae or Urbach-Wiethe disease) that is characterized by generalized thickening of skin, mucosae and certain viscera.

Provided are methods for generally detecting tumor-associated changes in the expression level in genes, such as changes in ECM1 expression levels, in brush cytology samples. In one aspect, the protein or nucleic acid expression level, e.g., increased expression, of the ECM1 gene is indicative of a likelihood that a subject has squamous cell carcinoma. In another aspect, protein or nucleic acid expression level, e.g., increased expression, of ECM1 is compared to a standard and is indicative of a likelihood that the subject has squamous cell carcinoma. In yet another aspect, protein or nucleic acid expression levels, e.g., increased expression, of the ECM1 gene compared to a standard is indicative of a likelihood that the subject has a precancerous squamous cell disorder. In one aspect, nucleic acid expression level of ECM1 can be monitored over time, at repeated intervals, and the expression level, e.g., increased expression, of the ECM1 gene is indicative of progression of squamous cell neoplasia. In another aspect, nucleic acids expression level of ECM1 is compared to a standard and over-expression of ECM1 is indicative of a likelihood that the subject has a precancerous squamous cell disorder. In yet another aspect, a kit is provided for assessing the presence of cancer in a sample comprising a pair of primers that specifically hybridize to at least one non-degraded ECM1 nucleic acid sequences and reagents for real-time RT-PCR.

### VIII. Predictive Medicine

The present invention pertains to the field of predictive medicine in which diagnostic assays, prognostic assays, pharmacogenetics and monitoring clinical trials are used for prognostic (predictive) purposes to thereby detect a precancerous, cancerous or progression of a squamous cell cancer. Accordingly, one aspect relates to diagnostic assays for detecting gene expression of nucleic acid and/or protein, in the context of a sample (e.g., brush cytology sample) to thereby detect the likelihood that a subject has a precancerous squamous cell disorder, has squamous cell carcinoma, or to monitor the progression of a squamous cell neoplasia, associated with increased or decreased nucleic acid and/or protein expression.

1. Harvesting the Sample

In one aspect, the sample can be a biopsy sample or a small number of cells or a tissue sample removed for processing. Common examples of biopsy methods can include, but are not limited to, brush cytology, core needle biopsy, surgical biopsy, punch biopsy, shave biopsy, incisional/excisional biopsy and curettage biopsy.

A brush cytology method can utilize a brush to obtain a transepithelial specimen with cellular representation from each of the three layers: the basal, intermediate, and superficial layers. Unlike some cytology instruments, which collect only exfoliated superficial cells, the brush cytology sample can penetrate to the basement membrane, removing tissue from all three epithelial layers of the mucosa. The brush cytology can be performed with or without topical or local anesthetic. The brush cytology instrument or brush can have one or two cutting surfaces. Brushes with one surface can comprise a rod with perpendicular bristles. Brushes with two surfaces can comprise a flat end of the brush and a circular border of the brush. Either surface can be used to obtain the specimen.

Brush cytology samples can be utilized to routinely detect precancerous disorders and carcinomas. The diagnosis of a cancer can be, accordingly, made when a lesion is suspicious enough that it causes a health practitioner or other person skilled in the art to refer the lesion for further analysis. Thus, the brush cytology can be a method of detecting a precancerous squamous cell disorder, which can prevent the cancer from developing further, and it can be a method of identifying unsuspected cancers at early and treatable stages.

The brush cytology sample can provide a health practitioner or other person skilled in the art with a diagnostic screening test. In one aspect, a brush cytology sample can be obtained. Prior to obtaining the sample, it is preferable to rinse the subjects mouth with physiologic saline (pH 7.4) to remove any foreign debris that can be collected during the sample harvest. The mouth rinse can be saline solution or any commercially available mouth wash. Firm pressure with a brush can be applied to the area to be sampled. In some embodiments, a brush can be rotated in at least 20 brush strokes, where a single brush stroke is a forward to backward/backward to forward, a side to side or circular movement to obtain the sample. In some other embodiments, a first brush can be rotated in two to five brush strokes, to prime the surface, then the first brush is discarded. Then a second brush can be rotated in the same location in at least 20 brush strokes to obtain the sample. Little to no bleeding should result after the sample harvest. In another embodiment, the brush cytology sample can comprise squamous cells.

2. Diagnostic Assays

An exemplary method for detecting the presence or absence of nucleic acid or protein in a biological sample involves obtaining a sample from a subject, e.g., brush cytology sample, assaying the expression level (e.g., mRNA, cDNA or protein) of genes (e.g., B2M, CYP1B1, KRT17, IL8, ANXA2, or LAMC2) and comparing the expression levels to a standard to detect the likelihood the subject has a precancerous squamous cell disorder, squamous cell carcinoma or to monitor the progression of a neoplasia. A preferred method for detecting expression level of messenger ribonucleic acid (mRNA) or complementary deoxyribonucleic acid (cDNA) can use amplification and quantification of specific nucleic acids. Such polymerase chain reaction (PCR) methods can be referred to as: quantitative PCR (q-PCR), real-time PCR (q-PCR) and quantitative real-time PCR, see also U.S. Pat. No. 6,171,785, which can be modified and adapted for use by methods known to those of ordinary skill in the art.

Primers based on the nucleotide sequence of the genes can be used to detect transcripts corresponding to the gene(s). In some embodiments, a primer pair can be designed by utilizing primer design software, such as GenScript, Primer3, PRIDE and Primer Express. Commercial primers are also available for purchase corresponding to multiple locations throughout the gene. In an exemplary embodiment, the primers can be complementary to a mRNA sequence of at least 15 bases found at least 500 basepairs and preferably at least 1000 basepairs from the encoded 3′ ends of the transcripts, corresponding to the transcriptional start site. By specifying at least 500 basepairs and preferably at least 1000 basepairs from the encoded 3′ ends of the transcripts for amplification, the q-PCR can be biased toward detecting the expression levels of non-degraded mRNA without interference of degraded mRNA that can be extracted from dead cells or cells undergoing apoptosis.

Another embodiment for detecting RNA or DNA corresponding to a gene or protein can be with the use of a labeled nucleic acid probe capable of hybridizing to a mRNA or cDNA. A wide variety of conventional techniques are available, including mass spectrometry, chromatographic separations, 2-D gel separations, binding assays (e.g., immunoassays), competitive inhibition assays, one- and two-dimensional gels and sandwiched ELISA. Typical methodologies for RNA detection include mRNA extraction from a cell or tissue sample, followed by hybridization of a labeled probe, (e.g., a complementary polynucleotide) specific for the target RNA to the extracted RNA, and detection of the probe (e.g., Northern blotting), direct sequencing, gel electrophoresis, column chromatography, and quantitative PCR.

The term “sample” is intended to include tissues, cells and biological samples isolated from a subject (e.g., brush cytology sample), as well as tissues, cells and fluids present within a subject. That is, the detection method can be used to detect mRNA, protein, or cDNA in a sample in vitro as well as mRNA or protein in vivo. For example, in vitro techniques for detection of mRNA can include PCR, q-PCR, northern hybridizations and in situ hybridizations and standard hybridization to complementary nucleic acid that is detectable by immunoassay. In vitro techniques for detection of protein can include enzyme linked immunosorbent assays (ELISAs), western blots, immunoprecipitations and immunofluorescence. In vitro techniques for detection of cDNA can include Southern hybridizations, PCR, q-PCR. Furthermore, in vivo techniques for detection of protein can include introducing into a subject a labeled antibody. For example, the antibody can be labeled with a radioactive label whose presence and location in a subject can be detected by standard imaging techniques.

In one aspect, methods for detecting the likelihood that a subject has squamous cell carcinoma can involve obtaining a sample (e.g., brush cytology sample) from a subject, extracting nucleic acids from the sample, mRNA, or generating to cDNA from mRNA, assaying the nucleic assaying the nucleic acids for expression level of non-degraded nucleic acid sequences coding for production of B2M, CYP1B1, KRT17, IL8, ANXA2, or LAMC2 and wherein over-expression of the B2M, KRT17, IL8, or ANXA2 gene compared to a standard, or under-expression of the LAMC2 or CYP1B1 gene compared to a second standard is indicative of a likelihood that the subject has squamous cell carcinoma. Examples of a standard can be, but are not limited to, a non-cancer cells sample, brush cytology sample from a control subject and normal cells.

In another aspect, the methods can involve obtaining a control sample (e.g., non-cancer cells sample) from a subject, extracting nucleic acids from the sample, mRNA, or generating to cDNA from mRNA, assaying the nucleic acids for expression level of non-degraded nucleic acid sequences coding for production of B2M, CYP1B1, KRT17, IL8, ANXA2, or LAMC2 and wherein over-expression of the B2M, KRT17, IL8, or ANXA2 gene compared to a standard or under-expression of the LAMC2 or CYP1B1 gene compared to a second standard is indicative of a likelihood that the subject has a precancerous squamous cell disorder.

Also disclosed are kits for detecting the presence of expression of the genes, B2M, CYP1B1, KRT17, IL8, ANXA2, or LAMC2, in a sample. For example, the kit can comprise a pair of primers which specifically hybridize to at least one non-degraded nucleic acid sequences coding for production of B2M, CYP1B1, KRT17, IL8, ANXA2, or LAMC2 gene and reagents for real-time polymerase chain reaction (q-PCR). The kit can further comprise a brush to obtain a brush cytology sample and nucleic acid extraction reagents. Furthermore, the kit can comprise instructions for using the kit to detect protein or nucleic acids.

In certain embodiments, detection of the expression levels can involve the use of a probe/primer in a polymerase chain reaction (PCR) (see, e.g., U.S. Pat. Nos. 4,683,195 and 4,683,202), such as PCR or q-PCR. This method can include the steps of collecting a sample of cells from a subject (such as a brush cytology sample), extracting nucleic acids (e.g., cDNA generated from mRNA, mRNA or both) from the cells of the sample, contacting the nucleic acid sample with one or more primers which specifically hybridize to a gene under conditions such that hybridization and amplification of the gene (if present) occurs, and detecting the expression level of an amplification product, and comparing the expression level to a standard.

In other embodiments, intensity assessment in electrophoretic mobility can be used to identify expression level of genes or genes encoding a protein. For example, amplification of the cDNA generated from the mRNA can be performed and the reaction product can be measured and quantified by electrophoresis.

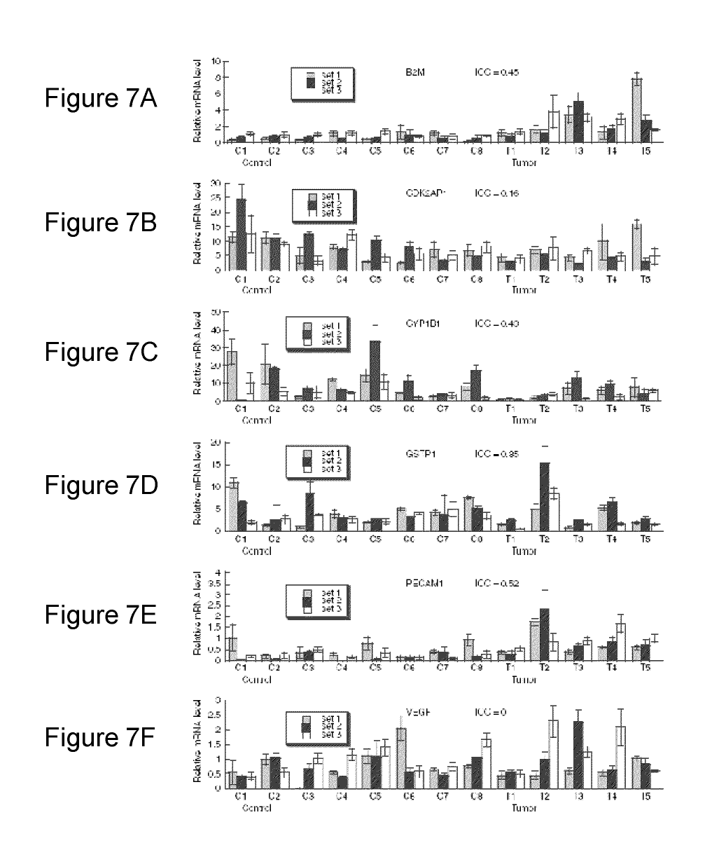

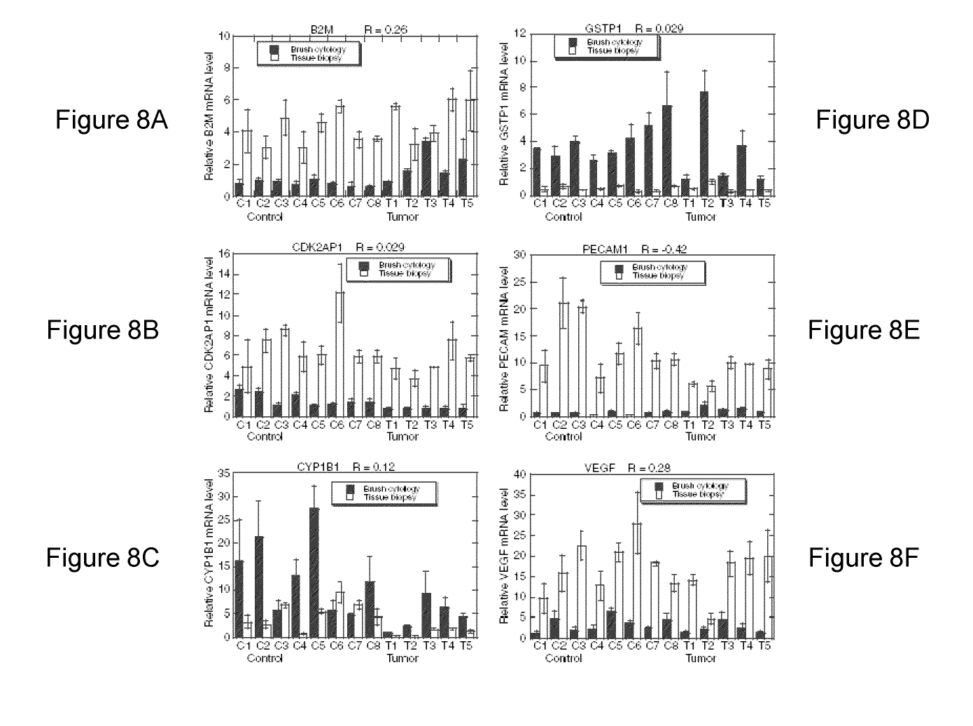

In one aspect, the results from assaying expression levels of tumor-associated genes, such as B2M, CYP1B1, KRT17, IL8, ANXA2, and LAMC2, can influence a treatment prescribed by a health care practitioner or other person of known skill in the art. Based on the analyzed expression levels of the tumor-associated genes, such as B2M, CYP1B1, KRT17, IL8, ANXA2, and LAMC2, additional assessments can be made to determine a treatment. The type of treatment options can be determined by those skilled in cancers. In some embodiments, repeated sampling can be done to monitor the progression of the squamous cell neoplasia over time when abberrant expression of tumor-associated genes is detected in initial assays. Some treatments can also include administering bioactive agents that act as activators of cytochrome p450 family members. These activators can potentially lead to increased expression of cytochrome p450 1B1 that is demonstrated in FIGS. 7C and 8C. Activators can also be administered to treat a squamous cell neoplasia by increasing expression of cytochrome p450.

3. Monitoring of the Progression of Neoplasia

Monitoring the cancer, e.g., squamous cell neoplasia, in a subject over time by assessing the expression of genes (e.g., B2M, CYP1B1, KRT17, IL8, ANXA2, and LAMC2) can monitor the progression of the squamous cell neoplasia. For example, the progression of squamous cell neoplasia over time can comprise an increase or decrease of gene expression levels or protein levels indicative of progression or inhibition of the neoplasia. Alternatively, the effectiveness of a treatment or the influence of agents (e.g., drugs) on the squamous cell neoplasia, can increase or decrease gene expression levels or protein levels. In such clinical trials, the expression levels of a gene or genes can be used as a “read out” of the progression or inhibition of the neoplasia.

For example, and not by way of limitation, genes, including genes and proteins encoded by the genes, that are altered by treatment with an agent (e.g., compound, drug or small molecule) can be identified. Thus, to study the effect of agents on gene-associated disorders (e.g., squamous cell carcinoma), for example, in a clinical trial, samples can be obtained and nucleic acids or proteins can be extracted and assayed for expression levels. The expression levels can be assayed by q-PCR, as described herein, or alternatively by measuring the amount of nucleic acid or protein produced, by one of the methods as described herein. In this way, the expression levels can be indicative of the physiological response of the neoplasia to the agent. Accordingly, the expression levels can be assayed before, and at various points during treatment with the agent.

In a preferred embodiment, a method is provided for monitoring squamous cell neoplasia in a human subject over time including the steps of (i) obtaining a brush cytology sample from a subject at a first time; (ii) extracting nucleic acids from cells in the sample; (iii) assaying said nucleic acids for the expression level of genes coding for the production of B2M, CYP1B1, KRT17, IL8, ANXA2, or LAMC2; and (iv) repeating the steps of obtaining a sample, extracting nucleic acids and assaying for expression levels of B2M, CYP1B1, KRT17, IL8, ANXA2, or LAMC2 at a later time, wherein increased expression of the B2M, KRT17, IL8, or ANXA2 gene at a later time or decreased expression of the LAMC2 or CYP1B1 gene at a later time is indicative of progression of neoplasia.

In another embodiment, squamous cell neoplasia in a human subject can be monitored over time in response to a treatment. A sample can be obtained, nucleic acids extracted from the sample, expression level of B2M, CYP1B1, KRT17, IL8, ANXA2, or LAMC2 can be assayed, a treatment can be administered, wherein the treatment is a bioactive agent that inhibits cytochrome p450 proteins, sampling from the subject can be repeated over time and the expression level of B2M, CYP1B1, KRT17, IL8, ANXA2, or LAMC2 at a later time is indicative of the response to the treatment.

4. Kits for Detecting Cancer

Also provided is a kit that can be used in the above methods. A kit for assessing cancer in a sample includes a means of detecting the expression levels of B2M, CYP1B1, KRT17, IL8, ANXA2, or LAMC2 genes in a sample. The present kit for cancer can include reagents used to make a diagnosis of cancer. Also, the present kit for cancer can comprise components used in publicly known kits, except that a means of detecting the expression level of genes associated with cancer (e.g., B2M, CYP1B1, KRT17, IL8, ANXA2, and LAMC2). Further, with the use of the kit for cancer, it can be possible to diagnose a subject as having cancer. Examples of cancer include, but are not limited to, squamous cell carcinoma. The kit can also be used to monitor progression of squamous cell neoplasias, as described above.

Herein, examples of detecting the presence of cancer by assaying expression levels cancer associated genes, can comprise:

(1) a pair of primers which specifically hybridize to at least one non-degraded nucleic acid sequences coding for production of B2M, CYP1B1, KRT17, IL8, ANXA2, or LAMC2 gene; and

(2) reagents for real-time polymerase chain reaction (q-PCR).

In additional embodiments the kit can comprise additional tools, reagents or instruction manuals. For example, the kit can comprise reagents for cDNA synthesis, a brush for obtaining a brush oral cytology sample from a subject. Also, the kit can comprise a nucleic acid extraction reagent to isolate nucleic acids from a sample.

In one embodiment, the kit can be a diagnostic kit for use in testing a sample. The kit can comprise one or more suitable pairs of primers for simultaneous or individual reverse transcription of different genes associated with cancer, such as B2M, CYP1B1, KRT17, IL8, ANXA2, or LAMC2, and optionally an appropriate calibrator mRNA in a single cDNA-synthesis reaction, standards or controls for q-PCR and/or standards or controls for B2M, CYP1B1, KRT17, IL8, ANXA2, or LAMC2 expression levels. The kit can be particularly useful for carrying out a variety of highly sensitive real-time PCRs (q-PCRs), thus allowing the quantification of expression levels of the tumor-associated genes, such as B2M, CYP1B1, KRT17, IL8, ANXA2, and LAMC2. For example, such a kit can include reagents for detecting expression levels of cancer associated genes, such as B2M, CYP1B1, KRT17, IL8, ANXA2, and LAMC2 (for example, primers and q-PCR reagents).

Another embodiment, the kit can contain instruction and reagents to simultaneously prime the reverse transcription of mRNA from more than one tumor associated genes in a single cDNA-synthesis reaction. Simultaneous quantification of genes by highly sensitive (reverse transcriptase PCR, RT-PCR) can reliably convert mRNA to cDNA by reverse transcription with reproducible efficiency.

In yet another embodiment, the kit can be used as a screening kit for presence of cancer in a sample or a series of samples. The kit can further be used as a method for monitoring the progression of a squamous cell neoplasia.

In one aspect, the kit can be used to determine expression levels of tumor-associated genes, such as B2M, CYP1B1, KRT17, IL8, ANXA2, and LAMC2, which can influence a treatment prescribed by a health care practitioner or other person skilled in the art. The type of treatment can be determined by those skilled in cancers and based on the results from the kit which analyzes expression levels of the tumor-associated genes, such as B2M, CYP1B1, KRT17, IL8, ANXA2, and LAMC2. In another embodiment, additional kits can be used over time to monitor the progression of the squamous cell neoplasia when over or under expression of B2M, CYP1B1, KRT17, IL8, ANXA2, and LAMC2 is detected. In another embodiment, multiple kits can be used over time to monitor the progression or inhibition of squamous cell neoplasia in response to a treatment with a bioactive agent by monitoring expression levels of B2M, CYP1B1, KRT17, IL8, ANXA2, or LAMC2.

### IX. Isolated Nucleic Acid and Proteins and Detection Methods

One aspect pertains to extracting nucleic acid molecules that either themselves are the nucleic acid sequences of interest (e.g., mRNA), or which encode the polypeptide, or fragments thereof. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

The term “extracted nucleic acid molecule” includes nucleic acid molecules which are separated from other molecules, such as other nucleic acid molecules or cellular debris which can be present within or associated with cells. For example, with regards to RNA, the term “isolated” includes RNA molecules which are separated from the other nucleic acids which are normally associated with RNA, such as DNA. Moreover, an “extracted” nucleic acid molecule, such as a cDNA molecule, can be substantially free of other cellular material, or culture medium when produced by recombinant techniques, or substantially free of chemical precursors or other chemicals when chemically synthesized. Nucleic acid molecules can be isolated from a cellular sample through means known by those skilled in the art, such as through cell lysis and precipitation and/or use of commercial reagents specialized in nucleic acid extraction.

A nucleic acid molecule, e.g., a nucleic acid molecule having the nucleotide sequence of the gene or a portion thereof, can be isolated using standard molecular biology techniques and the sequence information provided herein. Using all or a portion of the nucleic acid sequence of the gene as a hybridization probe, a gene or a nucleic acid molecule encoding a polypeptide can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

A nucleic acid can be amplified and quantified using mRNA or cDNA generated from mRNA as a template and appropriate oligonucleotide primers according to standard PCR amplification techniques and quantitative methods, such as q-PCR. The nucleic acids, moreover, can comprise non-degraded nucleic acid sequences coding for production of B2M, CYP1B1, KRT17, IL8, ANXA2, and LAMC2. In a more refined approach, cDNA copies of these mRNAs can be made using reverse transcriptase by methods known to those skilled in the art. A probe/primer can be generated to a specific portion of the genes to assay non-degraded mRNA (cDNA generated from mRNA), such as at least 500 basepairs and preferably at least 1000 basepairs from the encoded 3′ ends of the transcripts. The primers can be generated or purchased, as described above, such that they hybridize to at least about 10 to 12, preferably at least 15, found near at least 500 basepairs and preferably at least 1000 basepairs from the encoded 3′ ends of the transcripts.

In another embodiment, extracted nucleic acids can be at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes to a nucleic acid molecule corresponding to a nucleotide sequence of a gene.

When using PCR to quantitate expression levels, the product generated from the PCR can be at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length.

In one embodiment, proteins can be extracted from cells or tissue sources by an appropriate purification scheme using standard protein purification techniques. An “extracted” or “purified” protein or portion thereof is substantially free of cellular material or other contaminating proteins from the cell or tissue source from which the protein is derived, or substantially free from chemical precursors or other chemicals when chemically extracted. The language “substantially free of cellular material” includes preparations of protein in which the protein is separated from cellular components of the cells from which it is isolated or recombinantly produced. In one embodiment, the language “substantially free of cellular material” can include preparations of protein having less than about 30% (by dry weight) of other proteins (also referred to herein as a “contaminating protein”), more preferably less than about 20% of other proteins, still more preferably less than about 10% of other proteins, and most preferably less than about 5% other proteins. When the protein or portion thereof is chemically extracted, it can also be substantially free of chemical used for extraction, i.e., chemical represent less than about 20%, more preferably less than about 10%, and most preferably less than about 5% of the volume of the protein preparation.

As used herein, a “portion” of the protein includes a fragment of the protein comprising amino acid sequences sufficiently homologous to or derived from the amino acid sequence of the protein, which include fewer amino acids than the full length proteins. Typically, portions of the protein can comprise a domain or motif with at least one activity of the protein. A portion of the protein can be a polypeptide which is, for example, 10, 25, 50, 100, 200 or more amino acids in length. Portions of the protein can be used as targets for developing agents which modulate expression levels of the protein.

The proteins or nucleic acid sequences can be detected by any method known to those of skill in the art. A wide variety of conventional techniques are available, including mass spectrometry, chromatographic separations, 2-D gel separations, binding assays (e.g., immunoassays), competitive inhibition assays, and so on. Any effective method in the art for measuring the present/absence, level or activity of a protein or nucleic acid sequence is included. It is within the ability of one of ordinary skill in the art to determine which method would be most appropriate for measuring a specific protein or nucleic acid sequence. Thus, for example, a ELISA assay may be best suited for use in a physician's office while a measurement requiring more sophisticated instrumentation may be best suited for use in a clinical laboratory. Regardless of the method selected, it is important that the measurements be reproducible.

Quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another. In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In other preferred embodiments, the level of the proteins or nucleic acid sequences can be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the molecule. Standard protocols and data analysis are used to determine the marker concentrations from the assay data.

One embodiment for detecting RNA or DNA corresponding to a gene or protein can be with the use of a labeled nucleic acid probe capable of hybridizing to a mRNA or cDNA. Suitable probes for use in the diagnostic assays are described herein. A preferred agent for detecting protein is an antibody capable of binding to protein, preferably an antibody with a detectable label. Antibodies can be polyclonal, or more preferably, monoclonal. An intact antibody, or a fragment thereof (e.g., Fab or F(ab′)₂) can be used.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling can include detection of a primary antibody using a fluorescently labeled secondary antibody and end-labeling of a DNA probe with biotin such that it can be detected with fluorescently labeled streptavidin. The term “sample” is intended to include tissues, cells and biological samples isolated from a subject (e.g., brush cytology sample), as well as tissues, cells and fluids present within a subject. That is, the detection method can be used to detect mRNA, protein, or cDNA in a sample in vitro as well as mRNA or protein in vivo. For example, in vitro techniques for detection of mRNA can include PCR, q-PCR, northern hybridizations and in situ hybridizations. In vitro techniques for detection of protein can include enzyme linked immunosorbent assays (ELISAs), western blots, immunoprecipitations and immunofluorescence. In vitro techniques for detection of cDNA can include Southern hybridizations, PCR, q-PCR (e.g., as described in L. Cseke, et al., Handbook of Molecular and Cellular Methods in Biology and Medicine, 2^{nd} Ed., CRC Press, 2004). Furthermore, in vivo techniques for detection of protein can include introducing into a subject a labeled antibody. For example, the antibody can be labeled with a radioactive label whose presence and location in a subject can be detected by standard imaging techniques.

Measurement of the relative amount of an RNA or protein molecule can be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a gene may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

The proteins or nucleic acid sequences can be detected by any method known to those of skill in the art. Primers based on the nucleotide sequence of the genes can be used to detect transcripts corresponding to the gene(s). In some embodiments, a primer pair can be designed by utilizing primer design software, such as GenScript, Primer3, PRIDE and Primer Express. Commercial primers are also available for purchase corresponding to multiple locations throughout the gene. In an exemplary embodiment, the primers can be complementary to an mRNA sequence of at least 15 bases found at least 500 basepairs and preferably at least 1000 basepairs from the encoded 3′ ends of the transcripts, corresponding to the transcriptional start site. By specifying at least 500 basepairs and preferably at least 1000 basepairs from the encoded 3′ ends of the transcripts for amplification, the q-PCR can be biased toward detecting the expression levels of non-degraded mRNA without interference of degraded mRNA that can be extracted from dead cells or cells undergoing apoptosis.

### EXAMPLES

This invention is further illustrated by the following examples which should not be construed as limiting. The following experiments were performed to demonstrate various aspects of the invention.

### Example 1

### Materials and Methods

### Oral Carcinogenesis

Dibenzo[a,I]pyrene was applied orally at a level of 0.025 nm three times a week for 33 weeks to produce floor of the mouth and lateral border of tongue tumors in Golden Syrian Hamster (Mesocricetus auratus). Five of 12 animals developed oral squamous cell carcinoma (OSCC) detectable by gross inspection. These were later verified histologically. The average cross-sectional area of the lesions in these five was 3.2 mm². The first samples were taken from these five hamsters 1 month after the end of the carcinogen exposure (week 37). This was to insure that the observed gene expression changes were due to longterm changes in the tissue and not directly due to the presence of 0.0025 nM dibenz[a,I]pyrene. Eight hamsters treated identically but never exposed to dibenzo[a,I]pyrene were used as the source of control tissue. All procedures were carried out within the guidelines of the Animal Research Committee at the University of Illinois at Chicago.

### Cell and Tissue Acquisition

For brush cytology, a Cytosoft′ brush (Cytology Brush, Medical Packaging Corp., Camarillo, Calif., USA) was used to harvest oral keratinocytes from the mucosa, between 2:00 and 3:30 PM, on three consecutive weeks (weeks 37, 38, and 39). Twenty back and forth brushing motions were used. No trauma to the mucosa was noted. Brush oral cytology was applied to oral carcinoma and non-oral carcinoma sites. On the 40^{th} week, normal and tumor-bearing mucosa was surgically removed following asphyxiation with bottled carbon dioxide.

### Histopathology to Identify Oral Cancer

Tissues were processed, embedded, and sectioned at 5 um. Sections were stained using hematoxylin and eosin using an automated autostainer (Leica Microsystems, Bannokburn, Ill., USA) and evaluated using standard criteria.

### Immunohistochemistry

Cells were fixed in 2.5% formalin overnight then subjected to immunofluorescent staining using pancytokeratin-specific antibodies, clones: AE-1 and AE-3 (ab961) (AbCam, Cambridge, UK) as directed. The Ventana HX system (Ventana, Yokohama, Japan) was used to perform the immunofluorescent staining according to the manufacturer's protocol with standard enzymatic antigen retrieval. Tissue was treated identically except it was fixed in 10% formalin overnight and imbedded in paraffin prior to sectioning.

### RNA Extraction

Following brush cytology cell collection the brush was immersed in Trizol (Invitrogen, Carlsbad, Calif., USA), vortexed and then frozen at) 70° C. On thaw, the sample was vortexed, and then subjected to standard RNA isolation, followed by DNAse 1 treatment with the Aurum Total RNA Mini-kit as described by the manufacturer (Bio-Rad, Hercules, Calif., USA). cDNA synthesis was performed with ⅓ of the total sample of RNA, using random hexamers and Superscript III RT enzyme (Invitrogen). A similar process was used for the isolation of RNA from tissue, except mechanical homogenization was required in Trizol (Invitrogen).

Quantitative Real-Time q-PCR

Quantitative real-time q-PCR was carried out using the iCycler iQ (Bio-Rad) and SYBR Green fluorescence to detect double-stranded DNA. Values were normalized to the best controls, succinate dehydrogenase complex A (SDHA) and glyceraldehyde-3-phosphate dehydrogenase (GAPD) for brush cytology samples and cyclophilin A (PPIA) and beta-actin (ACTB), for tissue and brush cytology samples together. The quality of the RNA was judged to be satisfactory based on the fact that q-PCR with PPIA primer sets with different product sizes (120, 150, and 182 nucleotides) all gave similar results. Negative controls were without reverse transcriptase for cDNA synthesis. Amplicon sizes for primer pair products were validated using standard q-PCR with agarose gel ethidium bromide visualization. The results are reported as mean values from 3 to 6 separate samples. All PCR runs included a reference cDNA to allow the comparison of expression levels of samples tested at different times. Primer sets used included: forward primer hamster B2M (3′ AGTTTGTACCCACTGCGACTGA 5′) (SEQ ID NO.: 9); reverse primer hamster B2M (3′ TGCTGCTGTGTGCATAGACTGA 5′) (SEQ ID NO.: 10); forward primer human B2M (3′ TGTGCTCGCGCTACTCTCTCTTT 5′) (SEQ ID NO.: 11); reverse primer human B2M (3′ATGTCGGATGGATGAAACCCAGAC 5′) (SEQ ID NO.: 12); forward primer hamster CYP1B1 (3′ GAATCCATGCGCTTCTCCAGCTTT 5′) (SEQ ID NO.: 13); reverse primer hamster CYP1B1 (3′ TCCAGGAATCGGGCTGGATCAAAT 5′) (SEQ ID NO.: 14); forward primer human CYP1B1 (3′ GCCTCATTATGTCAACCAGGTCCA 5′) (SEQ ID NO.: 15); and reverse primer human CYP1B1 (3′ AAGCCAGGTAAACTCCAAGCACCT 5′) (SEQ ID NO.: 16).

Determination of Endogenous Controls for mRNA Levels of Brush Oral Cytology Harvested RNA

Direct analysis for RNA concentrations in brush cytology samples was impractical because of the low amounts (estimated at 20-200 ng), so the identification of reference genes to control for the mRNA levels was of great importance, see Table 1 (Sample 1 and 2 from Patient 1 and Sample A, B and C from Patient 2). Potential housekeeping genes for this purpose were identified based on their constant expression in many tissues or on consistent levels in normal and tumor tissue of the gastrointestinal tract based on data contained at the SAGEmap site of the Cancer Genome Anatomy Project.

Of the candidates, cDNA sequences for four (ACTB, GAPD, PPIA, and SDHA) were available in the Syrian Golden Hamster database, and a fifth, GSTP1, was added based on our observation that its expression was similar on average in tumor and normal oral mucosa (see FIGS. 8A-8F). We determined the expression level of these genes in brush cytology samples from eight examples of normal tissue and 10 examples of tumor tissue. We used the NORMFINDER program to identify the optimal control(s). This program determined which gene(s) varied minimally in expression levels when compared to average expression of the other potential reference genes. For tumor and control brush cytology samples (as in FIGS. 7A-7F) the geometric mean of the SDHA and GAPDH levels was identified as an optimal internal standard. Analogously, for tumor and control RNA from cytology and tissue biopsy samples together (as in FIGS. 8A-8F), the geometric mean of ACTB and PPIA levels was an optimal control.

TABLE 1
Proportion of undergraded Human beta
actin mRNA in different samples.

| | Sample 1 | Sample 2 | Sample A | Sample B | Sample C |
|---|---|---|---|---|---|
| Product-5′ | 0.0072 | 0.042 | 0.016 | 1.8 | 1 |
| Product-3′ | 0.45 | 1.2 | 0.186 | 1.8 | 3 |
| 5′/3′ | 0.016 | 0.034 | 0.086 | 1 | 0.33 |

### Statistical Analysis

The data presented are mean±SD unless otherwise stated. For statistical comparison of RNA levels between the control and tumor groups the Student's t-test was used. Results were considered statistically significant if the two-tailed P-values were <0.05. Analysis of variance (ANOVA) was used for the determination of the intraclass correlation (ICC) for repeated tests on the same hamster (FIGS. 7A-7F).

### Example 2

### Experimental Results

### Reliability of Quantitation of Brush Cytology Sample RNA

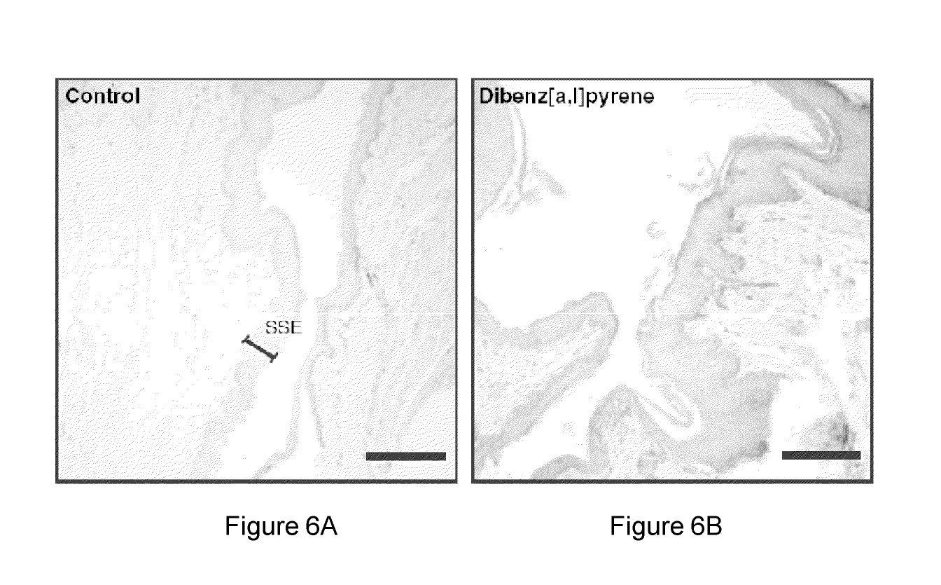

One month after the end of the dibenzo[a,I]pyrene exposure, brush cytology samples were harvested on three consecutive weeks from diseased and control unexposed hamsters (FIGS. 6A and 6B). RNA was purified and subjected to real-time q-PCR analysis (FIGS. 7A-7F). We used these two sources of cells (tumor epithelium and control mucosa) to increase the probability that specific RNA expression levels would vary among the different animals. The bar graphs in FIGS. 7A-7F show the measured level for each RNA of interest and allows an analysis of the reliability of the methodology described here. In addition to the tumor-associated genes, expression of the endothelial cell marker PECAM1 was also measured. The ICC was calculated as a measure of the degree of similarity between measurements carried out at different times for the same animal. It is compared to the degree of similarity of measurements for the different animals. While there was substantial lack of similarity for the weekly measurements on the same animal for some mRNAs, for three there was a relatively large ICC (FIGS. 7A-7F), verifying that there was substantial reproducibility in the measurement method. Nevertheless, it was clear that multiple samples would be necessary for the greatest accuracy. We also note that for three of six mRNAs (B2M, CYP1B1, and PECAM1), there were significant differences in expression levels in tumor vs. control samples (see Table 2).

TABLE 2
Comparison of mRNA levels in control vs. tumor
in samples acquired by different methods

Method of cell acquisition

| Gene | Brush cytology | Tissue biopsy |
|---|---|---|
| B2M | | |
| Control | 1.08 ± 0.111 | 4.00 ± 0.322 |
| Tumor | 2.59 ± 0.446 | 4.92 ± 0.576 |
| P-value | 0.00271 | 0.158 |
| CDK2AP1 | | |
| Control | 1.55 ± 0.168 | 7.11 ± 0.820 |
| Tumor | 0.709 ± .0847 | 5.31 ± 0.632 |
| P-value | 0.0643 | 0.0149 |
| CYP1B1 | | |
| Control | 13.2 ± 2.89 | 4.99 ± 0.96 |
| Tumor | 4.65 ± 1.42 | 1.22 ± 0.35 |
| P-value | 0.0154 | 0.0123 |
| GSTP1 | | |
| Control | 3.99 ± 0.419 | 0.50 ± 0.06 |
| Tumor | 2.98 ± 0.750 | 0.51 ± 0.14 |
| P-value | 0.862 | 0.941 |
| PECAM1 | | |
| Control | 0.447 ± 0.0781 | 13.3 ± 0.1.88 |
| Tumor | 1.10 ± 0.0202^{a} | 8.01 ± 0.938 |
| P-value | 0.00129 | 0.0572 |
| VEGF | | |
| Control | 3.34 ± 0.478^{a} | 15.3 ± 2.84 |
| Tumor | 2.48 ± 0.497 | 17.6 ± 2.12 |
| P-value | 0.381 | 0.520 |

^{a}A two-tailed Student's t-test was used to compare the statistical significance of the difference in mRNA levels of control mucosa and tumor.

Comparison of mRNA Levels in Brush Cytology and Tissue Biopsy Samples

It was then tested whether tumor-associated changes in the level of a specific mRNA in brush cytology samples would also be observed in RNA from tissue biopsies from the same animals. One week after the last cytologic sample was taken, the animals were killed and tissue from tumor and normal areas was taken by dissection to produce a tissue biopsy sample. To allow a comparison of RNA from all four sample types, RNA quantities were normalized to internal standards, PPIA, and BACT. An average for the three brush cytology samples is represented in the bar graph and is plotted next to the value obtained from the RNA from surgically biopsied tissue from the same animal (FIGS. 8A-8F). Surprisingly, the results were to some degree dependent on the sampling method. First, we note that there was minimal correlation between relative mRNA levels in brush cytology samples and surgical biopsy samples from the same animal (FIGS. 8A-8F 3). Secondly, in the same figure it is demonstrated that the levels of specific mRNAs depend on the sample type. Thirdly, only one of six genes, CYP1B1 showed a change in expression with tumor formation in the surgically excised tissue (Table 1). Specifically, CYP1B1 showed increased expression in the early timepoints and decreased expression in the later timepoints. In contrast, CYP1B1 and two other genes showed changes in the brush cytology samples with tumor formation. Brush cytology mRNA quantitation was reproducible but different from tissue biopsy mRNA. One simple explanation would be that brush cytology RNA was derived from different cells than that of the tissue biopsy RNA.

### Brush Cytology Sample RNA was Highly Enriched for Epithelial Markers

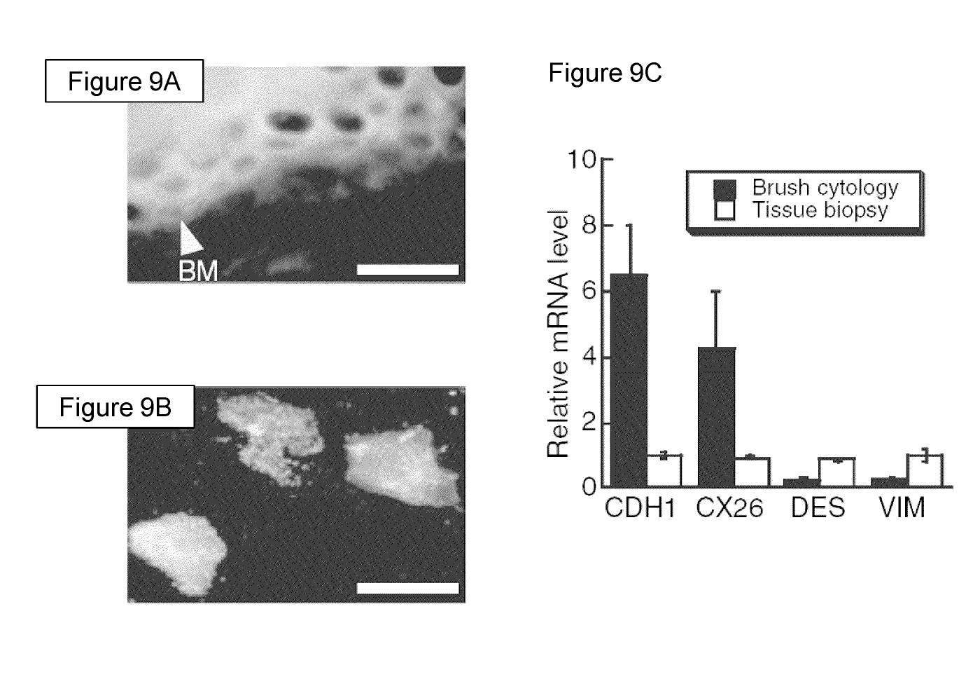

To determine the identity and purity of brush cytology cells we subjected normal tissue sections to immunofluorescence analysis of epithelial cytokeratins. A control experiment showed high levels of expression in the epithelium but not in the dermis (located below the basement membrane) of an immunostained section of biopsied tissue (FIG. 9A). In the brush cytology sample over 95% of cells contained high levels of these proteins (FIG. 9B). Further, RNA from five different brush cytology samples from control hamsters was compared to RNA from tissue biopsy samples from the same hamsters. In the brush cytology sample RNA epithelial cell markers, E-cadherin and connexin-26 (CADH1 and CX26), were enriched, while desmin (DES), a muscle cell marker, and vimentin (VIM), a marker for mesenchymally derived cells, were depressed (FIG. 9C). This is consistent with the brush cytology sample being greatly enriched for mucosal epithelial cells compared to the tissue biopsy cells.

### Example 3

### Methods

### Subjects

Samples were collected from former and current tobacco and betel users who presented with oral lesions necessitating a biopsy to rule out malignancy in the Oral and Maxillofacial Surgery Clinic and the Otolaryngology Clinic in the University of Illinois Medical Center. Diagnoses were determined by biopsy and histopathological analysis unless noted. Three normal samples were from lesion free patients with no pathology detectable by biopsy. Excluded were subjects with prior history of head and neck cancer chemotherapy or irradiation treatment. All subjects provided consent to participate in accordance with guidelines of the Institutional Review Board of the University of Illinois at Chicago.

### Quantitative Real-Time PCR

RNA was collected from the brush directly in Trizol and frozen until further purified using the RNAeasy Mini Kit (Qiagen, Valencia, Calif.) with removal of DNA using column purification. The cDNA synthesis was as described earlier with approximately 70 nanograms RNA per reaction and also oligo dt primers. Quantitative real time PCR was carried out using the iCycler iQ (Bio-Rad, Hercules, Calif.) and SYBR Green fluorescence to detect double stranded DNA. 20 Values were normalized to the geometric mean of the controls. GAPD, RPLPO and RPL4 were selected as internal controls as these mRNAs showed similar relative expression levels in each sample. 21-23 Primers for these mRNAs and those to detect, B2M, CYP1B1, KRT17, SPINK5 and ECM1 were designed to give products of approximately 100 bases and are included in the supplemental data section.

### Statistical Analysis and Class Prediction

Analysis of variance (ANOVA) was used for the determination of the intraclass correlation coefficient (ICC) for mRNA measurements from two separate samples of the same oral site in a subject for SPINK5, and also for ECM1. For class comparison, due to the nonnormal distribution of B2M, CYP1B1 and KRT17 expression levels the Wilcoxon test was used to determine the statistical significance of the differences. A gene expression based classifier to differentiate OSCC versus non-malignant oral tissue using RNA from cytology samples was developed and tested using BRB array. After log 2 transformation of the normalized expression levels of mRNAs, B2M, CYP1B1 and KRT17, the data was imported into the program to undergo simultaneous testing of six different algorithms, including compound covariate predictors (CCP), k—nearest neighbor, nearest centroid, support vector machine (SVM), and diagonal linear discriminant analysis (LDA). These algorithms use different aspects of the data to perform classification, with k—nearest neighbor and nearest centroid both being nonlinear and nonparametric methods. Leave-one-out-cross-validation (LOOCV) was used to simultaneously develop a classifier using an algorithm and to test the misclassification rate. Predictive performance was compared to the prevalence of the more common sample type, nonmalignant, and an error rate assuming 100% assignment to that larger group

Reliability of RNA Quantitation from Brush Cytology

Reliability of gene expression measurements of RNA from brush cytology of human subjects was demonstrated. Duplicate brush cytology samples from one site were obtained from six subjects with no obvious pathology and two additional subjects with OSCC. RT-PCR analysis revealed three optimal housekeeping genes (data not shown). Based on the absorbance at 260 nm there was as much as a 20 variation in levels of the total RNA content between replicates (data not shown).

In order to detect the expression of individual mRNAs in the duplicate samples, genes known to show high expression levels in oral mucosa, but vary in expression in surgical samples from oral cancer and other diseases, were analyzed. For example, Spink5 mRNA, encoding a serpin, is highly expressed in epithelium of the oral mucosa and skin and can show variable expression depending on inflammation levels.

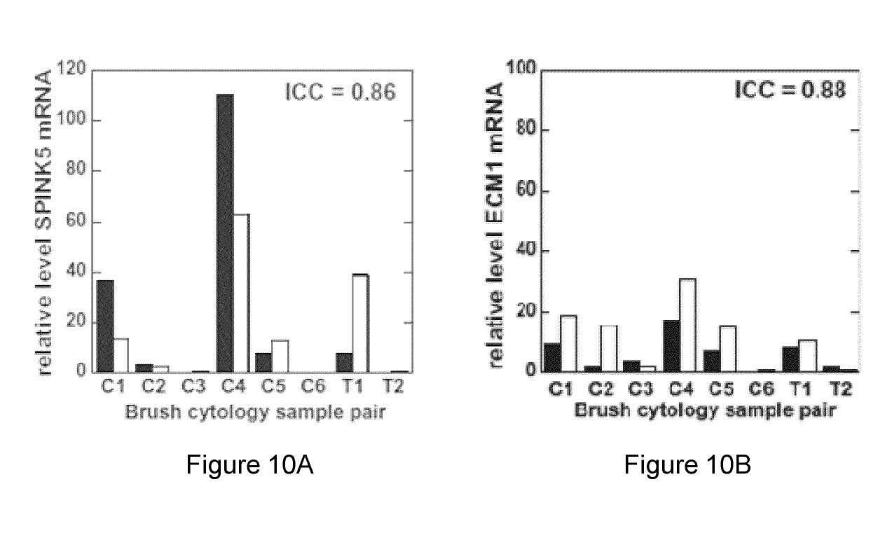

Relative expression levels of SPINK5 were calculated in relationship to mRNA levels for the three housekeeping genes. Despite the variability in total RNA in each brush cytology pair, as measured by the level of housekeeping gene expression, a strong intraclass correlation between expression level in the two samples, (FIG. 10A and FIG. 10B), was found. Shown in FIG. 10A and FIG. 10B are two brush cytology samples that were consecutively harvested from the same site in patients C1-C8 without lesions, and T1 and T2 with OSCC. RT-PCR analysis of levels of SPINK5 mRNA (FIG. 10A) and ECM1 mRNA (FIG. 10B) revealed an intraclass correlation coefficient (ICC) of 0.81 and 0.86 respectively for SPINK5 and ECM1 mRNA between the first and second samplings.

### Differential Expression

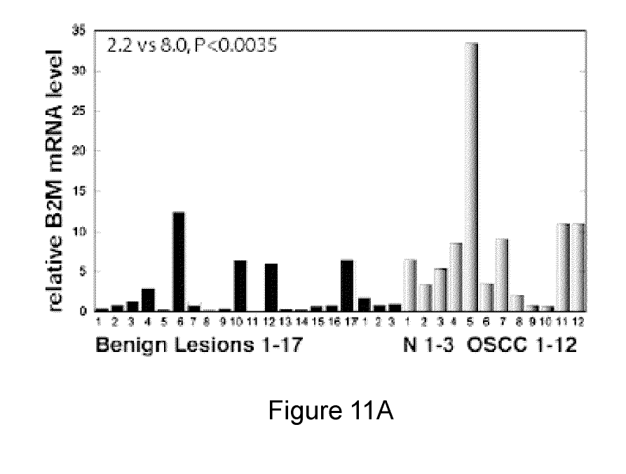

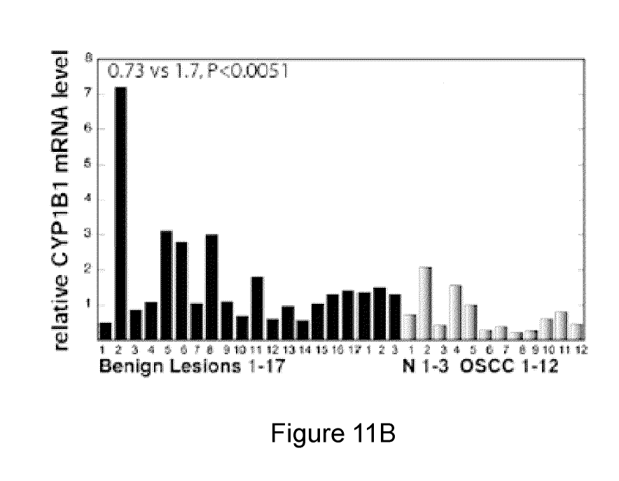

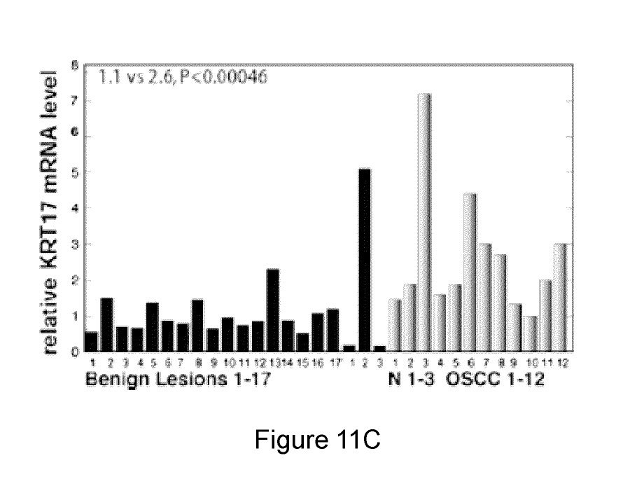

Risk factor for the 12 patients with OSCC, the 17 patients with nonmalignant lesions and the three additional controls with no are shown in Table 3 and 4. All subjects had a history of usage of the oral carcinogens, tobacco, or betel. CYP1B1 and B2M, genes that we had earlier shown to be differentially expression in a hamster model of oral cancer induction by a tobacco and environmental carcinogen, dibenz[a,I]pyrene, and KRT17 were shown to be enriched in surgically obtained tissue from OSCC, and versus normal tissue in a recent brush cytology study. FIG. 11A and FIG. 11C reveal expression of both B2M and KRT17, respectively, were enriched in the OSCC samples. Each bar represents the relative mRNA level for B2M (FIG. 11A), CYP1B1 (FIG. 11B), KRT17 (FIG. 11C), measured by RT-PCR and normalized to the geometric mean of GAPD, RPLO and RPL4 levels. The samples are grouped according to the diagnosis. In the left corner is the mean level of expression of the OSCCs versus the benign lesions and normals and the significance of this difference based on the Wilcoxon test.

TABLE 3
Tumor samples: Patient data and OSCC details.

| | | | | Tobacco/ | TNM | |
|---|---|---|---|---|---|---|
| Sample | Site^{a} | Sex | Age | Betel^{b} | Classification | Grade |
| OSCC1 | UG | M | 45 | Tob/Bet | T1N0M0 | Gr 1 |
| OSCC2 | LG | M | 46 | F-Tob | T4aN0M0 | Gr 2 |
| OSCC3 | UG | M | 65 | F-Tob/ | Ta1N1M0 | Gr 1 |
| | | | | F-Bet | | |
| OSCC4 | FOM | M | 55 | Tob | T2N0M0 | Gr 2 |
| OSCC5 | Bu | M | 64 | Tob | T4aN0M0 | Gr 2 |
| OSCC6 | LG | M | 38 | Tob | T4aN2bM0 | Gr 2 |
| OSCC7 | FOM | M | 64 | Tob | T1N0M0 | Gr 2 |
| OSCC8 | T | M | 81 | Tob | T2N0M0 | Gr 1 |
| OSCC9 | Bu | M | 53 | Tob | T2N0M0 | Gr 2 |
| OSCC10 | FOM-T | M | 60 | Tob | T4aN0M0 | Gr 1 |
| OSCC11 | T | M | 64 | F-Tob | T1N0M0 | Gr 3 |
| OSCC12 | P | M | 61 | Tob | T3N0M0 | Gr 2 |

^{a}T—tongue; P—palate; LG/UG—lower/upper gingival; FOM—floor of mouth,; Bu—buccal mucosa.
^{b}Tob—Tobacco user; Bet—betel nut user, if former user than F-Tob or F-Bet.

TABLE 4
Pathological appearance of nonmalignant lesions
and description of nonpathological controls.

| | Site^{a} | Sex | Age | Tobbaco/betel | Lesion diagnosis^{b} |
|---|---|---|---|---|---|
| BL1 | UG | M | 49 | Tob | Leukoplakia |
| BL2 | T | F | 40 | Tob | Leukoplakia |
| BL3 | LG | M | 22 | Tob | Ameloblastoma |
| BL4 | T | F | 68 | Bet | Ulceration |
| BL5 | LG | M | 70 | Tob/Bet | Leukoplakia |
| BL6 | UG | M | 53 | Tob | Leukoplakia |
| BL7 | LM | M | 61 | F-Tob | Mucocele |
| BL8 | Bu | F | 54 | Tob | Lipoma |
| BL9 | P | F | 62 | Tob | fibroma |
| BL10 | Bu | M | 22 | Tob | Lichen planus |
| BL11 | UG | F | 83 | F-Tob | Pyongenic granuloma |
| BL12 | P | F | 56 | Tob | Leukoplakia |
| BL13 | UG | M | 43 | Tob | Leukoplakia |
| BL14 | T | M | 25 | Tob | Granular cell tumor |
| BL15 | Bu | M | 59 | Tob | Epulis fisularum |
| BL16 | FOM | F | 50 | Tob | Sialolithiasis |
| BL17 | T | M | 61 | Tob | Traumatic Ulcer |
| C1 | UG | M | 42 | Tob | Normal |
| C2 | UG | M | 45 | Tob | Normal |
| C3 | UG | M | 70 | F-Tob | Normal |

^{a}T—tongue; P—palate; LG/UG—lower/upper gingival; FOM—floor of mouth,; Bu—buccal mucosa; Tob—Tobacco user; Bet—betel nut user, if former user than F-Tob or F-Bet.
^{b}No dysplasia observed except in BL6, which showed mild dysplasia.

The results in FIG. 11A, FIG. 11B and FIG. 11C are consistent with the potential for these mRNAs to serve as markers for this disease. CYP1B1 (FIG. 11B) was expressed at decreased levels in OSCC brush cytology samples, also making it a potential OSCC marker.

### Classification of OSCC vs Non-Tumor Samples

In order to test the utility of quantitation of these three, or a subset of these three, mRNAs in RNA from brush cytology to classify OSCC and non-malignancies, a small group of supervised classification algorithms was tested. Of these, K-nearest neighbor, nearest centroid, SVM and LDA all showed an 81% rate of correct classification as derived by external LOOCV based on the level of all three mRNAs. This compared favorably to the baseline classification rate of 63% (20 non-malignancies/32 total samples) if one selected all samples as non-malignancies, the more prevalent sample type.

### Example 4

### Materials and Methods

### Clinical Sampling

Samples were collected from former and current tobacco and betel users that presented with oral lesions necessitating a biopsy to rule out malignancy in the Oral and Maxillofacial Surgery Clinic and the Otolaryngology Clinic in the University of Illinois Medical Center. Samples from this earlier study, along with those from additional patients, make up the 14 OSCC samples and 20 nonmalignant controls of the classifier training set. Nineteen additional RNAs from brush cytology samples of the validation set were collected from 6 OSCC and 13 nonmalignant lesions from the same number of patients in those clinics. All diagnoses were verified by histopathological examination of surgically obtained tumor tissue for OSCCs and scalpel biopsy material for nonmalignancies. All subjects provided consent to participate in accordance with guidelines of the Institutional Review Board of the University of Illinois at Chicago.

### Brush Cytology

Brush cytology was performed taking care to minimize tissue damage, samples were immediately placed in Trizol, mixed and frozen and the RNA purified. The Agilent Bioanalyzer 2100 was used to analyze the quality of a subset of RNA samples which indicated significant degradation as shown earlier with buccal RNA from brush cytology. Two hundred nanograms of total RNA was subjected to two rounds of linear amplification using the GeneChip® Expression 3′-Amplification Two-Cycle cDNA Synthesis kit (Affymetrix Inc., Santa Clara, Calif.) and biotin labeled, fragmented, antisense RNA hybridized to an Affymetrix Human Genome U133 Plus 2.0 microarray. Once completed, arrays were processed according to the manufacturer's protocol and scanned using the GeneChip® Scanner 3000 (Affymetrix). The Microarray Suite Version 5 (Affymetrix) was used to generate signal values.

### Gene Target Selection

Based on reviews on a wide range of head and neck cancer studies, and original studies that focused on OSCC, 70 genes that showed differential expression with oral pharyngeal cancer in multiple independent studies were counted. Due to the limited amount of RNA from each sample, in some cases less than 150 ng, mRNAs with maximal average expression levels in the epithelium were the focus. Results of the global gene expression analysis provided insight to the identification of these highly expressed genes, those that could be assayed using RT-PCR with SYBR green detection so that 60 ng total RNA supplied sufficient cDNA for about 250 assays. In a preliminary test, mRNAs with background corrected average hybridization levels to the U133 Plus 2.0 microarray of >32 units were found to fulfill the criteria. Importantly for individual mRNAs, a strong correlation between average signal strength across all the arrays versus the signal strength in the RT-PCR assays that followed (data not shown) was found. Over 1500 genes on the array had signal above 32 units on average, including 43 of the 70 head and neck cancer genes previously identified. Twenty-one genes were tested on samples from OSCCs and nonmalignant control patients. In addition to these, mRNAs from 6 other genes were tested and chosen due to differential expression in OSCC.

### RT-PCR

RNA from brush cytology was converted to cDNA and quantitative real time PCR was carried out using the iCycler iQ (Bio-Rad, Hercules, Calif.) and SYBR Green fluorescence. Values were normalized to the geometric mean of the controls, GAPD, RPLPO and RPL4. Primers for these mRNAs, and those to detect the target mRNAs, were designed using Primer Express to give products of approximately 100 bases. See Table 5.

TABLE 5
Sequences used as PCR primers.

| Gene | Sense | Antisense |
|---|---|---|
| ANAX1 | 5′-ACCAGAAGCTATCCACAACTTCGC-3′ | 5′-CACTTCACGATAGCTGTGAGGCAT-3′ |
| | (SEQ ID.: 17) | (SEQ ID.: 18) |
| ANXA2 | 5′-TGCTGATCGGCTGTATGACTCCAT-3′ | 5′-ACTTCACTGCGGGAGACCATGATT-3′ |
| | (SEQ ID.: 19) | (SEQ ID.: 20) |
| ALD9 | 5′-ACCAGCTGAAGACTCTGTGGGA-3′ | 5′-CAGCGTGGCCATGTCAATAGGTTT-3′ |
| | (SEQ ID.: 21) | (SEQ ID.: 22) |
| ARPC1 | 5′-AGTGGTAGTGTTTCAGAGGCCAGA-3′ | 5′-TGGGTACATGGCGTCTGTTTCTCA-3′ |
| | (SEQ ID.: 23) | (SEQ ID.: 24) |
| C11ORF48 | 5′-GTGCCAACATTACACTGTCAGGGA-3′ | 5′-TTCACTAGTCCTGGCTGGCTTTGA-3′ |
| | (SEQ ID.: 25) | (SEQ ID.: 26) |
| C20ORF3 | 5′-ATTTGGGAAGCTCCTTGCACTTGG-3′ | 5′-AAAGAAGAACTTTGAGGCCGAAGG-3′ |
| | (SEQ ID.: 27) | (SEQ ID.: 28) |
| CAV1 | 5′-GCTGAGTAAAGCACTTGCAACCGT-3′ | 5′-TCTTTCTGGGCAAAGGGATGCTTG-3′ |
| | (SEQ ID.: 29) | (SEQ ID.: 30) |
| CEACAM1 | 5′-GGCCTCTGCTAAGGTGTATTTGGT-3′ | 5′-TTAGCACCAGTGCAGCTTTCTAGC-3′ |
| | (SEQ ID.: 31) | (SEQ ID.: 32) |
| CSTB1 | 5′-TCGTACACCTGCGAGTGTTCCAAT-3′ | 5′-AAGGGCCTTGTCCAAAGTCAGGAT-3′ |
| | (SEQ ID.: 33) | (SEQ ID.: 34) |
| CXCL1 | 5′-AAATCCACCTGACCAGAAGGGAGG-3′ | 5′-TCTGCAGCTGTGTCTCTCTTTCCT-3′ |
| | (SEQ ID.: 35) | (SEQ ID.: 36) |
| EMP1 | 5′-TAAGAACAGAGTGCCTGCATTCCC-3′ | 5′-AGCTCGTCTACCATCTGACTAGGT-3′ |
| | (SEQ ID.: 37) | (SEQ ID.: 38) |
| IL6 | 5′-CTCATTCTGCGCAGCTTTAAGGAG-3′ | 5′-CAACAATCTGAGGTGCCCATGCTA-3′ |
| | (SEQ ID.: 39) | (SEQ ID.: 40) |
| IL8 | 5′-ACAAGTCCTTGTTCCACTGTGCCT-3′ | 5′-TCACTGTGAGGTAAGATGGTGGCT-3′ |
| | (SEQ ID.: 41) | (SEQ ID.: 42) |
| ISG15 | 5′-AGCACCGTGTTCATGAATCTGC-3′ | 5′ACAGCCTTTATTTCCGGCCCTT-3′ |
| | (SEQ ID.: 43) | (SEQ ID.: 44) |
| KRT4 | 5′-GGGCAGAGATCGAGAACATCAAGA-3′ | 5′CCAGGAGCAGCAAGATCATCTCTACCA-3′ |
| | (SEQ ID.: 45) | (SEQ ID.: 46) |
| KRT13 | 5′GGAGTGCCAGAACCAAGAGTACAA-3′ | 5′AGGCACTAGAAGTCGTGGTGGTAACAG-3′ |
| | (SEQ ID.: 47) | (SEQ ID.: 48) |
| LAMC2 | 5′GATGGGTCACTGAACACCTATTGCAC-3′ | 5′-AGGATTCCCAAGCTGTCTCGTGTT-3′ |
| | (SEQ ID.: 49) | (SEQ ID.: 50) |
| LAPT4 | 5′-ACCATCTGCTGACTGTTCTTGTGG-3′ | 5′-ATGCAGCGCCAAACACATCCATTC-3′ |
| | (SEQ ID.: 51) | (SEQ ID.: 52) |
| MAL | 5′-TTTGAGTTTGACGCAGCCTACCAC-3′ | 5′-GAGAACACCACGGCAATGTTT-3′ |
| | (SEQ ID.: 53) | (SEQ ID.: 54) |
| MMPI | 5′-ATGCAACTCTGACGTTGATCCCAG-3′ | 5′-GACTGCACATGTGTTCTTGAGCTG-3′ |
| | (SEQ ID.: 55) | (SEQ ID.: 56) |
| MMP12 | 5′-AGACAGGTTCTTCTGGCTGAAGGT-3′ | 5′-TTCAATGCCAGATGGCAAGGTTGG-3′ |
| | (SEQ ID.: 57) | (SEQ ID.: 58) |
| PPL | 5′-TCCCAACCATCATTCACCCTGA-3′ | 5′-TGTTGCTGGGAGTGTACAGGAA-3′ |
| | (SEQ ID.: 59) | (SEQ ID.: 60) |
| SCEL1 | 5′-TGGAAGCTCTAACACTGGAGCCAA-3′ | 5′-CTCCTGATATCCATCCTTGGGTGA-3′ |
| | (SEQ ID.: 61) | (SEQ ID.: 62) |
| TGM3 | 5′-ACTTCTCCTGCAACAAGTTCCCTG-3′ | 5′-TGTTGTCCAAGTTTGTACGGGAGG-3′ |
| | (SEQ ID.: 63) | (SEQ ID.: 64) |

### Statistical Analysis, Class Comparison and Class Prediction

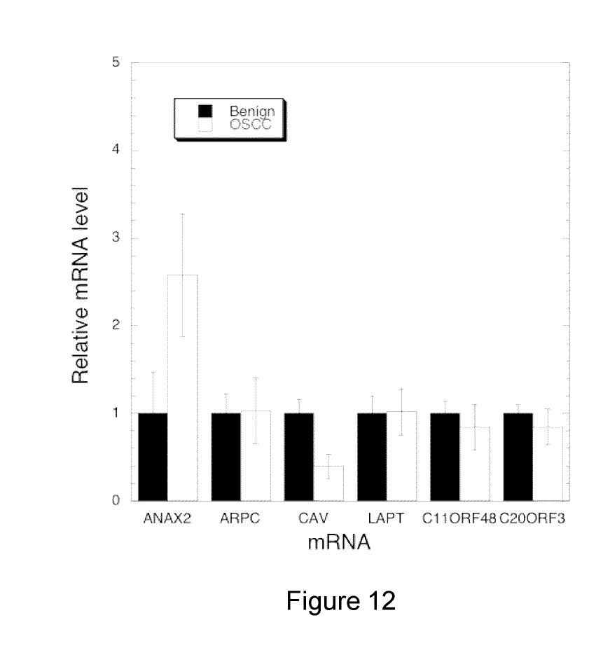

The class comparison function of BRB-Array Tools 3.9 (two-sample t-test with random variance model) allowed the determination of mRNA of genes that discriminate between RNA from cytology samples from OSCC and nonmalignant lesions with a maximum allowed proportion of false positive genes of 0.1. The Student's t test was used to determine if the 6 OSCC genes identified in only a single published study, showed differential expression in 6 OSCC and 7 nonmalignant samples (FIG. 12).

A gene expression based classifier was generated using the class predictor function of BRB Array tools as described. Briefly, normalized mRNA levels for the 22 targets were log 2 transformed and entered for each sample of the training set. The program used 6 separate algorithms to generate optimized predictors while simultaneously performing Leave-One-Out-Cross-Validation of the generated classifiers. In the end Support Vector Machines generated the classifier. This classifier was tested on the 19 samples of the validation set again using BRB-Array Tools.

### Example 5

### Experimental Results

Brush oral cytology samples were obtained from 34 patients, 14 with oral squamous cell carcinoma and 20 with nonmalignant oral lesions (see Table 6) all tobacco or betel users. Diagnoses were verified with histopathology of biopsy and surgical tissue. Based on the number of genes interrogated and an observed within class standard deviation of 0.7 and cutoff for differential expression of 2 fold between classes, a minimum of 11 samples from each group was estimated to define a classifier with a tolerance of 0.10. Yields for the RNA from brush cytology varied between 0.15 to 4 μg per sample of column purified RNA, placing limits on the RNA analyses. A pilot study was performed to determine global gene expression levels of RNA from brush cytology of 3 OSCC and 3 benign lesions via hybridization to Affymetrix Human Genome U133 Plus 2.0 microarrays. Because of the limited quantity of many RNA from brush cytology samples, a decision was made to take advantage of the high sensitivity, accuracy and dynamic range of RT-PCR analysis of mRNA levels of known OSCC associated genes to examine gene expression in this study for the remainder of the study.

TABLE 6
Training Set: Patient data and OSCC details tumor samples.

| | | | | Tobacco/ | TNM | |
|---|---|---|---|---|---|---|
| Sample^{a} | Site^{b} | Sex | Age | Betel^{c} | Classification | Grade |
| OSCC 1 | UG | M | 45 | Tob and | T1NOMO | Gr 1 |
| | | | | Bet | | |
| OSCC 2 | LG | M | 46 | F-Tob | T4aN0M0 | Gr 2 |
| OSCC 3 | UG | M | 65 | F-Tob | Ta1N1M0 | Gr 1 |
| OSCC 4 | FOM | M | 55 | Tob | T2N0M0 | Gr 2 |
| OSCC 5 | Bu | M | 64 | Tob | T4aN0M0 | Gr 2 |
| OSCC 6 | LG | M | 38 | Tob | T4aN2bM0 | Gr 2 |
| OSCC 7 | FOM | M | 64 | Tob | T1N0M0 | Gr 2 |
| OSCC 8 | T | M | 81 | Tob | T2N0M0 | Gr 1 |
| OSCC 9 | Bu | M | 53 | Tob | T2N0M0 | Gr 2 |
| OSCC 10 | FOM- | M | 60 | Tob | T4aN0M0 | Gr 1 |
| | TM | | | | | |
| OSCC 11 | T | M | 64 | F-Tob | T1N0M0 | Gr 3 |
| OSCC 12 | P | M | 61 | Tob | T3N0M0 | Gr 2 |
| OSCC 56 | UG | M | 84 | F-Tob | T4aNoMo | Gr 2 |
| OSCC 63 | LG | M | 85 | Tob | T4bN3M1 | Gr 1 |

^{a}OSCC1-OSCC12.
^{b}T—tongue; P—palate; LG/UG—lower/upper gingival; FOM—floor of mouth,; Bu—buccal mucosa.
^{c}Tob—Tobacco user; Bet—betel nut user, if former user than F-Tob or F-Bet.

Using RT-PCR, a classifier for OSCC based on the 70 known OSCC associated genes along with B2M, a gene we have shown to be linked to OSCC in brush oral cytology samples was derived. Genes that were expressed at higher levels as defined in the methods, and that were amenable to RT-PCR with SYBR green detection were focused on. BRB-array tools were used to perform a class comparison between the two sample sets in order to determine genes that were differentially expressed. Six genes showed differential expression in OSCC versus control brush cytology RNA samples based on producing a fold change of over 2× and an FDR of below 0.1 (Table 7). FDR is the false discovery rate and a measure of the probability that the data point is differentially expressed. Number of samples tested refers to number of tumor samples (T) and nonmalignant samples (N) that provided expression data for these mRNAs. Interestingly, one of these mRNAs, ANXA1 showed an increase in expression in OSCC samples, while earlier studies that examined RNA isolated from surgically obtained tissue, had indicated a decrease.

TABLE 7
RT-PCR analysis of gene expression using RNA from
brush cytology of OSCC versus benign lesions.

| | | | Fold-change | | Number of |
|---|---|---|---|---|---|
| | Parametric p- | | in mRNA | | Samples |
| | value | FDR | level | GENE | Tested |
| 1 | 0.0003012 | 0.00663 | 0.21 | LAMC2 | 13T 24N |
| 2 | 0.0009211 | 0.0101 | 4.08 | ANXA2 | 14T 24N |
| 3 | 0.0014582 | 0.0107 | 2.49 | KRT17 | 14T 24N |
| 4 | 0.0054184 | 0.0298 | 9.43 | IL8 | 14T 23N |
| 5 | 0.0098977 | 0.0435 | 4.46 | ANXA1 | 14T 24N |
| 6 | 0.017282 | 0.0585 | 3.33 | ECM1 | 14T 24N |
| 7 | 0.018623 | 0.0585 | 4.02 | B2M | 14T 24N |
| 8 | 0.0908235 | 0.225 | 0.43 | IL6 | 10T 15N |
| 9 | 0.0922038 | 0.225 | 3.76 | MAL | 13T 21N |
| 10 | 0.1030077 | 0.227 | 1.83 | MMP12 | 11T 22N |
| 11 | 0.1568689 | 0.314 | 3.18 | CXCL | 14T 24N |
| 12 | 0.1817851 | 0.333 | 2.32 | TGM3 | 13T 22N |
| 13 | 0.2025812 | 0.343 | 2.24 | EMP | 11T 17N |
| 14 | 0.2489573 | 0.378 | 2.42 | MMP1 | 10T 21N |
| 15 | 0.2765658 | 0.378 | 2.14 | Spink5 | 12T 18N |
| 16 | 0.3184763 | 0.378 | 1.57 | SCEL | 12T 18N |
| 17 | 0.3228818 | 0.378 | 1.39 | LAMC2 | 12T 17N |
| 18 | 0.3414761 | 0.378 | 2.32 | KRT4 | 10T 18N |
| 19 | 0.3435379 | 0.378 | 2.03 | KRT13 | 12T 22N |
| 20 | 0.5283724 | 0.554 | 1.62 | ISG15 | 13T 22N |
| 21 | 0.8653252 | 0.865 | 0.96 | ALD9 | 11T 17N |
| 22 | 0.2991023 | 0.422 | 0.44 | CSTB1 | 11T 15N |

FDR—false discovery rate.

Changes in expression of 6 genes, LAPT, C20orf3, ARPC1, C11orf48, ANXA2 and CAV1 were also tested. The genes were arbitrarily chosen among those reported to be differentially expressed on the RNA level in tissue from OSCC and nonmalignant or normal sites in a single study. A preliminary analysis of RNA from oral cytology samples from 7 OSCC lesion and 8 nonmalignant lesions was done (FIG. 12). Of the mRNAs analyzed those from the ANXA2 and CAV 1 genes showed a statistically significant, or near significant changes in levels based on the student T test (Table 6).

In order to perform class prediction for OSCC using the RNA from cytology samples, BRB array tools were used to perform Leave-One-Out-Cross-Validation, simultaneously testing 7 classifiers for their ability to differentiate OSCC from nonmalignant samples based on the levels of the 20 tested genes. Four out of six methods were found. Compound covariate predictor, Diagonal Linear Discriminant Analysis, 1—nearest neighbor, showed approximately 89% accuracy in identifying these samples. Support Vector Machines generated a classifier that showed the highest level of success in correct classification of OSCC and nonmalignant RNA from cytology samples in the training set with 92% accuracy. The Support Vector Machines was tested and generated a classifier on an independent validation set of 18 samples of RNA from cytology of 6 OSCC lesion and 12 nonmalignant lesions (Tables 8 and 9). The classifier made two errors, incorrectly classifying one tumor and one nonmalignant lesion, but was correct 89% of the time. The specificity was 88% and the sensitivity 84%.

TABLE 8
Validation Set: Tumor samples patient data and OSCC details.

| Sample | Site^{a} | Sex | Age | Tobacco/Betel^{b} | TNM | Grade |
|---|---|---|---|---|---|---|
| OSCC 81 | RMF | M | 61 | Tob | T4aN0M0 | Gr 2 |
| OSCC 102 | LG | M | 46 | Tob | T4bN3M0 | Gr 1 |
| OSCC 127 | FOM | M | 52 | Tob | T1N0M0 | Gr 1 |
| OSCC 204 | T | F | 76 | Tob | T1N0M0 | Gr 1 |
| OSCC 213 | T | F | 55 | Tob | T1N0M0 | Gr 3 |
| OSCC 216 | FOM | M | 67 | Tob | T2N0M0 | Gr 3 |

^{a}T—tongue; SP—soft palate; HP—Hard palate; LG/UG—lower/upper gingival; FOM—floor of mouth; Bu—buccal mucosa, LM—lip mucosa.
^{b}Tob—Tobacco user; Bet—betel nut user, if former user than F-Tob or F-Bet.

Results of RT-PCR for quantitation of ANXA2, when tested against the entire training set of samples are included in Table 9. CAV1 mRNA analysis was not pursued due to low mRNA levels.

TABLE 9
Validation Set: Pathological appearance of nonmalignant
lesions and description of nonpathological controls.

| Sample | Site^{a} | Sex | Age | Tobacco/Betel^{b} | Diagnosis |
|---|---|---|---|---|---|
| BL66 | SP | M | 93 | F-Tob | Pleomorphic adenoma |
| BL93 | UG | F | 63 | Tob | KCOT |
| BL115 | Bu | M | 22 | Bet/Tob | Submucous Fibrosis |
| BL120 | LG | M | 55 | Tob | Leukoplakia |
| BL130 | LG | M | 32 | F-Tob | Ameloblastoma |
| BL142 | Bu | M | 53 | Tob | Lichen planus |
| BL151 | HP | M | 88 | Tob. | Nicotinic stomatitis |
| BL158 | T | M | 65 | Tob | Lichen planus, |
| | | | | | candidiasis |
| BL29 | Bu | F | 66 | Tob | Hyperplastic foliat |
| | | | | | papilloma |
| BL225 | Bu | F | 56 | Tob | Lichen planus |
| BL2337 | LM | M | 52 | Tob | Salivary gland tumor |
| BL242 | SP | F | 39 | Tob | Papilloma |
| BL278 | gingiva | M | 57 | Tob | Verruciform |
| | | | | | xanthoma, |
| | | | | | mild dysplasia |

^{a}T—tongue; SP—soft palate; HP—Hard palate; LG/UG—lower/upper gingival; FOM—floor of mouth; Bu—buccal mucosa, LM—lip mucosa.
^{b}Tob—Tobacco user; Bet—betel nut user, if former user than F-Tob or F-Bet.

While the present invention has been described in terms of specific methods, structures, and devices it is understood that variations and modifications will occur to those skilled in the art upon consideration of the present invention. For example, the methods and compositions discussed herein can be utilized beyond the embodiments disclosed. As well, the features illustrated or described in connection with one embodiment can be combined with the features of other embodiments. Such modifications and variations are intended to be included within the scope of the present invention. Those skilled in the art will appreciate, or be able to ascertain using no more than routine experimentation, further features and advantages of the invention based on the above-described embodiments. Accordingly, the invention is not to be limited by what has been explicitly shown and described.

All publications and references are herein expressly incorporated by reference in their entirety. The terms “a” and “an” can be used interchangeably, and are equivalent to the phrase “one or more” as utilized in the present application. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A method for detecting the likelihood that a subject has a presence of or a risk for development of a cancerous oral squamous cell disorder, comprising:
obtaining a brush cytology sample from a subject;
assaying expression levels of beta-2 microgobulin (B2M) and at least one additional gene in the brush cytology sample; and
comparing expression level of B2M with a standard and comparing the at least one additional gene with a second standard, wherein over-expression of B2M compared with the standard and differential expression of the at least one additional gene compared with the second standard is indicative of a likelihood that the subject has the presence of or the risk for development of the cancerous oral squamous cell disorder.

2. The method of claim 1, wherein the brush cytology sample comprises oral squamous cells.

3. The method of claim 1, wherein the step of assaying expression levels further comprises amplifying and quantifying expression of the B2M gene and the at least one additional gene by real time polymerase chain reaction (RT-PCR) using primers complementary to an mRNA sequence of at least 15 bases found near the 5′ ends of the B2M gene and the at least one additional gene.

4. The method of claim 1, wherein the step of obtaining the sample further comprises at least 20 brush strokes.

5. The method of claim 1, wherein the step of obtaining the sample further comprises taking 2 to 5 initial brush strokes to prime the surface, followed by at least 20 brush strokes to obtain the sample.

6. The method of claim 1, wherein the additional gene is selected from cytochrome p450 1B1 (CYP1B1), keratin 17 (KRT17), interleukin 8 (IL8), annexin A2 (ANXA2), and laminin gamma-2 (LAMC2).

7. The method of claim 1, wherein the step of assaying expression levels of B2M and the additional gene occurs simultaneously.

8. A method for detecting the likelihood that a subject has a presence of or a risk for development of a cancerous oral squamous cell disorder, comprising detecting protein or nucleic acid expression level of beta-2 microgobulin (B2M) and at least one additional gene in a brush cytology sample from the subject, wherein over-expression of the B2M gene compared to a standard together with differential expression of the additional gene compared to a second standard is indicative of a likelihood that the subject has a presence of or a risk for development of a cancerous oral squamous cell disorder.

9. The method of claim 8, wherein the step of obtaining the sample further comprises at least 20 brush strokes.

10. The method of claim 8, wherein the step of obtaining the sample further comprises taking 2 to 5 initial brush strokes to prime the surface, followed by at least 20 brush strokes to obtain the sample.

11. The method of claim 8, wherein the additional gene is selected from cytochrome p450 1B1 (CYP1B1), keratin 17 (KRT17), interleukin 8 (IL8), annexin A2 (ANXA2), and laminin gamma-2 (LAMC2).

12. The method of claim 1, wherein the step of detecting protein or nucleic acid expression level of B2M and the additional gene occurs simultaneously.

13. A method for monitoring squamous cell neoplasia in a human subject over time, comprising:
obtaining a brush cytology sample from a subject at a first time, assaying expression level of beta-2 microgobulin (B2M) and at least one additional gene, and
repeating the steps of obtaining a sample and assaying for expression levels of B2M and at least one additional gene at a later time,
wherein over-expression of the B2M gene at a later time or differential expression of the additional gene compared at a later time is indicative of progression of neoplasia.

14. A kit for assessing the presence of oral cancer in a sample comprising:
a pair of primers which specifically hybridize to a non-degraded nucleic acid sequence encoding beta-2 microglobulin (B2M);
a pair of primers which specifically hybridize to at least one additional non-degraded nucleic acid sequence; and
reagents for real-time polymerase chain reaction (RT-PCR).

15. The kit of claim 14 used according to the method of claim 1.

16. The kit of claim 14 used according to the method of claim 7.

17. The kit of claim 14, further comprising a brush to obtain a brush cytology sample.

18. The kit of claim 14, further comprising a nucleic acid extraction reagent.

19. The kit of claim 14, wherein the pair of primers for the additional non-degraded nucleic acid sequence specifically hybridizes to a nondegraded mRNA or cDNA of at least one of cytochrome p450 1B1 (CYP1B1), keratin 17 (KRT17), interleukin 8 (IL8), annexin A2 (ANXA2), or laminin gamma-2 (LAMC2).

20. A method of assaying gene expression in a tissue sample from a subject comprising detecting nucleic acid or protein expression level of beta-2 microgobulin (B2M) and at least one additional gene or protein of interest in the sample, wherein over-expression of B2M compared to a standard together with differential expression of the additional gene or protein of interest compared to a second standard is indicative of a likelihood that the subject has a presence of or a risk for development of a cancerous squamous cell disorder.

21. The method of claim 20, wherein the step of detecting nucleic acid or protein expression level comprises simultaneously detecting nucleic acid or protein expression levels of B2M and the additional gene or protein.

22. The method of claim 20, wherein the step of detecting nucleic acid expression levels comprises detecting nucleic acids that are at least 500 nucleotides in length.

23. The method of claim 20, wherein the tissue sample comprises dead or dying cells.

24. The method of claim 23, wherein the step of detecting nucleic acid expression level comprises detecting nucleic acids that are partially degraded and less than about 500 nucleotides in length.

25. The method of claim 20, wherein the tissue sample is from a mouth, lip, tongue, cheek lining, gingiva, palate, skin, esophagus, vagina and cervix of the subject.


SEQUENCE LISTING (64 sequences)

SEQ ID NO: 1 - DNA, 987 bp, Homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 2 - DNA, 5160 bp, Homo sapiens
aaaacccggaggagcgggatggcgcgctttgactctggagtgggagtgggagcgagcgcttctgcgactccagttgtgagagccgcaagggcatgggaattgacgccactcaccgacccccagtctcaatctcaacgctgtgaggaaacctcgactttgccaggtccccaagggcagcggggctcggcgagcgaggcacccttctccgtccccatcccaatccaagcgctcctggcactgacgacgccaagagactcgagtgggagttaaagcttccagtgagggcagcaggtgtccaggccgggcctgcgggttcctgttgacgtcttgccctaggcaaaggtcccagttccttctcggagccggctgtcccgcgccactggaaaccgcacctccccgcagcatgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataagaagcaagaggcaagctgaaattttagaaatattcacatcttcggagatgaggagtaaaattcagtttttttccagttcctcttttgtgctgcttctcaattagcgtttaaggtgagcataaatcaactgtccatcaggtgaggtgtgctccatacccagcggttcttcatgagtagtgggctatgcaggagcttctgggagatttttttgagtcaaagacttaaagggcccaatgaattattatatacatactgcatcttggttatttctgaaggtagcattctttggagttaaaatgcacatatagacacatacacccaaacacttacaccaaactactgaatgaagcagtattttggtaaccaggccatttttggtgggaatccaagattggtctcccatatgcagaaatagacaaaaagtatattaaacaaagtttcagagtatattgttgaagagacagagacaagtaatttcagtgtaaagtgtgtgattgaaggtgataagggaaaagataaagaccagaaattcccttttcaccttttcaggaaaataacttagactctagtatttatgggtggatttatccttttgccttctggtatacttccttacttttaaggataaatcataaagtcagttgctcaaaaagaaatcaatagttgaattagtgagtatagtggggttccatgagttatcatgaattttaaagtatgcattattaaattgtaaaactccaaggtgatgttgtacctcttttgcttgccaaagtacagaatttgaattatcagcaaagaaaaaaaaaaaagccagccaagctttaaattatgtgaccataatgtactgatttcagtaagtctcataggttaaaaaaaaaagtcaccaaatagtgtgaaatatattacttaactgtccgtaagcagtatattagtattatcttgttcaggaaaaggttgaataatatatgccttgtataatattgaaaattgaaaagtacaactaacgcaaccaagtgtgctaaaaatgagcttgattaaatcaaccacctatttttgacatggaaatgaagcagggtttcttttcttcactcaaattttggcgaatctcaaaattagatcctaagatgtgttcttatttttataacatctttattgaaattctatttataatacagaatcttgttttgaaaataacctaattaatatattaaaattccaaattcatggcatgcttaaattttaactaaattttaaagccattctgattattgagttccagttgaagttagtggaaatctgaacattctcctgtggaaggcagagaaatctaagctgtgtctgcccaatgaataatggaaaatgccatgaattacctggatgttctttttacgaggtgacaagagttggggacagaactcccattacaactgaccaagtttctcttctagatgattttttgaaagttaacattaatgcctgctttttggaaagtcagaatcagaagatagtcttggaagctgtttggaaaagacagtggagatgaggtcagttgtgttttttaagatggcaattactttggtagctgggaaagcataaagctcaaatgaaatgtatgcattcacatttagaaaagtgaattgaagtttcaagttttaaagttcattgcaattaaacttccaaagaaagttctacagtgtcctaagtgctaagtgcttattacattttattaagctttttggaatctttgtaccaaaattttaaaaaagggagtttttgatagttgtgtgtatgtgtgtgtggggtggggggatggtaagagaaaagagagaaacactgaaaagaaggaaagatggttaaacattttcccactcattctgaattaattaatttggagcacaaaattcaaagcatggacatttagaagaaagatgtttggcgtagcagagttaaatctcaaataggctattaaaaaagtctacaacatagcagatctgttttgtggtttggaatattaaaaaacttcatgtaattttattttaaaatttcatagctgtacttcttgaatataaaaaatcatgccagtatttttaaaggcattagagtcaactacacaaagcaggcttgcccagtacatttaaattttttggcacttgccattccaaaatattatgccccaccaaggctgagacagtgaatttgggctgctgtagcctatttttttagattgagaaatgtgtagctgcaaaaataatcatgaaccaatctggatgcctcattatgtcaaccaggtccagatgtgctataatctgtttttacgtatgtaggcccagtcgtcatcagatgcttgcggcaaaaggaaagctgtgtttatatggaagaaagtaaggtgcttggagtttacctggcttatttaatatgcttataacctagttaaagaaaggaaaagaaaacaaaaaacgaatgaaaataactgaatttggaggctggagtaatcagattactgctttaatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccattaccaaaataataaaaatcactttcataatcttgaaaaaa

SEQ ID NO: 3 - DNA, 1543 bp, Homo sapiens
ctctcctctccagcccttctcctgtgtgcctgcctcctgccgccgccaccatgaccacctccatccgccagttcacctcctccagctccatcaagggctcctccggcctggggggcggctcgtcccgcacctcctgccggctgtctggcggcctgggtgccggctcctgcaggctgggatctgctggcggcctgggcagcaccctcgggggtagcagctactccagctgctacagctttggctctggtggtggctatggcagcagctttgggggtgttgatgggctgctggctggaggtgagaaggccaccatgcagaacctcaatgaccgcctggcctcctacctggacaaggtgcgtgccctggaggaggccaacactgagctggaggtgaagatccgtgactggtaccagaggcaggccccggggcccgcccgtgactacagccagtactacaggacaattgaggagctgcagaacaagatcctcacagccaccgtggacaatgccaacatcctgctacagattgacaatgcccgtctggctgctgatgacttccgcaccaagtttgagacagagcaggccctgcgcctgagtgtggaggccgacatcaatggcctgcgcagggtgctggatgagctgaccctggccagagccgacctggagatgcagattgagaacctcaaggaggagctggcctacctgaagaagaaccacgaggaggagatgaacgccctgcgaggccaggtgggtggtgagatcaatgtggagatggacgctgccccaggcgtggacctgagccgcatcctcaacgagatgcgtgaccagtatgagaagatggcagagaagaaccgcaaggatgccgaggattggttcttcagcaagacagaggaactgaaccgcgaggtggccaccaacagtgagctggtgcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctgaggactcagctaccccggccggccacccaggaggcagggaggcagccgccccatctgccccacagtctccggcctctccagcctcagccccctgcttcagtcccttccccatgcttccttgcctgatgacaataaagcttgttgactcagctaaaaaaaaaaaaaaaaaaaaaaaaaaaaaaaaaaaaaaaaaaa

SEQ ID NO: 4 - DNA, 1605 bp, Homo sapiens
gaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 5 - DNA, 1385 bp, Homo sapiens
agtgtgaaatcttcagagaagaatttctctttagttctttgcaagaaggtagagataaagacactttttcaaaaatggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacactgaagaaagcccttacaggtcaccttgaggaggttgttttggctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaactaaacattcccttgatggtctcaagctatgatcagaagactttaattatatattttcatcctataagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgaaaaaaaaaaaaaaaaaaaaaaaaaaaaaa

SEQ ID NO: 6 - DNA, 1500 bp, Homo sapiens
gctcagcatttggggacgctctcagctctcggcgcacggcccagggtgaaaatgtttgccattaaactcacatgaagtaggaaatatttatatggatacaaaaggcacctgcatgggataatgtcaaatttcatagatactgctttgtgcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtacaaaaaaaaaaaaaaaaaaaaaaaaaaaaaa

SEQ ID NO: 7 - DNA, 3414 bp, Artificial Sequence
  ARTIFICIAL
cccctgcagcggagacagagactgagcggcccggcaccgccatgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactccggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgtcactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagaccctgtctattttgtggctcctgccaaatttcttgggaatcaacaggtgagctatggtcaaagcctgtcctttgactaccgtgtggacagaggaggcagacacccatctgcccatgatgtgattctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgatgccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacaacaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctcggcttcagggagtcagtgatcagtcctttcaggtggaagaagcaaagaggatcaaacaaaaagcggattcactctcaagcctggtaaccaggcatatggatgagttcaagcgtacacagaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatgggtatgtgaacccacaacccacaaccttccagctccatgctccagg

SEQ ID NO: 8 - DNA, 1853 bp, Artificial Sequence
  ARTIFICIAL
agaagagctggtcctgaagctcacaaccgtaacagccaccagacaagcttcagtggccggcccttcacatccagacttgcctgagaggacccacctctgagtgtccagtggtcagttgccccaggatggggaccacagccagagcagccttggtcttgacctatttggctgttgcttctgctgcctctgagggaggcttcacggctacaggacagaggcagctgaggccagagcactttcaagaagttggctacgcagctcccccctccccacccctatcccgaagcctccccatggatcaccctgactcctctcagcatggccctccctttgagggacagagtcaagtgcagccccctccctctcaggaggccacccctctccaacaggaaaagctgctacctgcccaactccctgctgaaaaggaagtgggtccccctctccctcaggaagctgtccccctccaaaaagagctgccctctctccagcaccccaatgaacagaaggaaggaatgccagctccatttggggaccagagccatccagaacctgagtcctggaatgcagcccagcactgccaacaggaccggtcccaagggggctggggccaccggctggatggcttcccccctgggcggccttctccagacaatctgaaccaaatctgccttcctaaccgtcagcatgtggtatatggtccctggaacctaccacagtccagctactcccacctcactcgccagggtgagaccctcaatttcctggagattggatattcccgctgctgccactgccgcagccacacaaaccgcctagagtgtgccaaacttgtgtgggaggaagcaatgagccgattctgtgaggccgagttctcggtcaagacccgaccccactggtgctgcacgcggcagggggaggctcggttctcctgcttccaggaggaagctccccagccacactaccagctccgggcctgccccagccatcagcctgatatttcctcgggtcttgagctgcctttccctcctggggtgcccacattggacaatatcaagaacatctgccacctgaggcgcttccgctctgtgccacgcaacctgccagctactgaccccctacaaagggagctgctggcactgatccagctggagagggagttccagcgctgctgccgccaggggaacaatcacacctgtacatggaaggcctgggaggatacccttgacaaatactgtgaccgggagtatgctgtgaagacccaccaccacttgtgttgccgccaccctcccagccctactcgggatgagtgctttgcccgtcgggctccttaccccaactatgaccgggacatcttgaccattgacatcagtcgagtcacccccaacctcatgggccacctctgtggaaaccaaagagttctcaccaagcataaacatattcctgggctgatccacaacatgactgcccgctgctgtgacctgccatttccagaacaggcctgctgtgcagaggaggagaaattaaccttcatcaatgatctgtgtggtccccgacgtaacatctggcgagaccctgccctctgctgttacctgagtcctggggatgaacaggtcaactgcttcaacatcaattatctgaggaacgtggctctagtgtctggagacactgagaacgccaagggccagggggagcagggctcaactggaggaacaaatatcagctccacctctgagcccaaggaagaatgagtcaccccagagccctagagggtcagatggggggaaccccaccctgccccacccatctgaacactcattacactaaacacctcttggaaaaaaaaaaaaaaaaaa

SEQ ID NO: 9 - DNA, 22 bp, Artificial Sequence
  ARTIFICIAL
agtttgtacccactgcgactga

SEQ ID NO: 10 - DNA, 22 bp, ARTIFICIAL
  ARTIFICIAL
tgctgctgtgtgcatagactga

SEQ ID NO: 11 - DNA, 23 bp, ARTIFICIAL
  ARTIFICIAL
tgtgctcgcgctactctctcttt

SEQ ID NO: 12 - DNA, 24 bp, ARTIFICIAL
  ARTIFICIAL
atgtcggatggatgaaacccagac

SEQ ID NO: 13 - DNA, 24 bp, Artificial Sequence
  ARTIFICIAL
gaatccatgcgcttctccagcttt

SEQ ID NO: 14 - DNA, 24 bp, ARTIFICIAL
  ARTIFICIAL
tccaggaatcgggctggatcaaat

SEQ ID NO: 15 - DNA, 24 bp, ARTIFICIAL
  ARTIFICIAL
gcctcattatgtcaaccaggtcca

SEQ ID NO: 16 - DNA, 24 bp, ARTIFICIAL
  ARTIFICIAL
aagccaggtaaactccaagcacct

SEQ ID NO: 17 - DNA, 24 bp, ARTIFICIAL
  ARTIFICIAL
accagaagctatccacaacttcgc

SEQ ID NO: 18 - DNA, 24 bp, ARTIFICIAL
  ARTIFICIAL
cacttcacgatagctgtgaggcat

SEQ ID NO: 19 - DNA, 24 bp, ARTIFICIAL
  ARTIFICIAL
tgctgatcggctgtatgactccat

SEQ ID NO: 20 - DNA, 24 bp, ARTIFICIAL
  ARTIFICIAL
acttcactgcgggagaccatgatt

SEQ ID NO: 21 - DNA, 22 bp, ARTIFICIAL
  ARTIFICIAL
accagctgaagactctgtggga

SEQ ID NO: 22 - DNA, 24 bp, ARTIFICIAL
  ARTIFICIAL
cagcgtggccatgtcaataggttt

SEQ ID NO: 23 - DNA, 24 bp, ARTIFICIAL
  ARTIFICIAL
agtggtagtgtttcagaggccaga

SEQ ID NO: 24 - DNA, 24 bp, ARTIFICIAL
  ARTIFICIAL
tgggtacatggcgtctgtttctca

SEQ ID NO: 25 - DNA, 24 bp, ARTIFICIAL
  ARTIFICIAL
gtgccaacattacactgtcaggga

SEQ ID NO: 26 - DNA, 24 bp, ARTIFICIAL
  ARTIFICIAL
ttcactagtcctggctggctttga

SEQ ID NO: 27 - DNA, 24 bp, ARTIFICIAL
  ARTIFICIAL
atttgggaagctccttgcacttgg

SEQ ID NO: 28 - DNA, 24 bp, ARTIFICIAL
  ARTIFICIAL
aaagaagaactttgaggccgaagg

SEQ ID NO: 29 - DNA, 24 bp, ARTIFICIAL
  ARTIFICIAL
gctgagtaaagcacttgcaaccgt

SEQ ID NO: 30 - DNA, 24 bp, ARTIFICIAL
  ARTIFICIAL
tctttctgggcaaagggatgcttg

SEQ ID NO: 31 - DNA, 24 bp, ARTIFICIAL
  ARTIFICIAL
ggcctctgctaaggtgtatttggt

SEQ ID NO: 32 - DNA, 24 bp, ARTIFICIAL
  ARTIFICIAL
ttagcaccagtgcagctttctagc

SEQ ID NO: 33 - DNA, 24 bp, ARTIFICIAL
  ARTIFICIAL
tcgtacacctgcgagtgttccaat

SEQ ID NO: 34 - DNA, 24 bp, ARTIFICIAL
  ARTIFICIAL
aagggccttgtccaaagtcaggat

SEQ ID NO: 35 - DNA, 24 bp, ARTIFICIAL
  ARTIFICIAL
aaatccacctgaccagaagggagg

SEQ ID NO: 36 - DNA, 24 bp, ARTIFICIAL
  ARTIFICIAL
tctgcagctgtgtctctctttcct

SEQ ID NO: 37 - DNA, 24 bp, ARTIFICIAL
  ARTIFICIAL
taagaacagagtgcctgcattccc

SEQ ID NO: 38 - DNA, 24 bp, ARTIFICIAL
  ARTIFICIAL
agctcgtctaccatctgactaggt

SEQ ID NO: 39 - DNA, 24 bp, ARTIFICIAL
  ARTIFICIAL
ctcattctgcgcagctttaaggag

SEQ ID NO: 40 - DNA, 24 bp, ARTIFICIAL
  ARTIFICIAL
caacaatctgaggtgcccatgcta

SEQ ID NO: 41 - DNA, 24 bp, ARTIFICIAL
  ARTIFICIAL
acaagtccttgttccactgtgcct

SEQ ID NO: 42 - DNA, 24 bp, ARTIFICIAL
  ARTIFICIAL
tcactgtgaggtaagatggtggct

SEQ ID NO: 43 - DNA, 22 bp, ARTIFICIAL
  ARTIFICIAL
agcaccgtgttcatgaatctgc

SEQ ID NO: 44 - DNA, 22 bp, ARTIFICIAL
  ARTIFICIAL
acagcctttatttccggccctt

SEQ ID NO: 45 - DNA, 24 bp, ARTIFICIAL
  ARTIFICIAL
gggcagagatcgagaacatcaaga

SEQ ID NO: 46 - DNA, 27 bp, ARTIFICIAL
  ARTIFICIAL
ccaggagcagcaagatcatctctacca

SEQ ID NO: 47 - DNA, 24 bp, ARTIFICIAL
  ARTIFICIAL
ggagtgccagaaccaagagtacaa

SEQ ID NO: 48 - DNA, 27 bp, ARTIFICIAL
  ARTIFICIAL
aggcactagaagtcgtggtggtaacag

SEQ ID NO: 49 - DNA, 26 bp, ARTIFICIAL
  ARTIFICIAL
gatgggtcactgaacacctattgcac

SEQ ID NO: 50 - DNA, 24 bp, ARTIFICIAL
  ARTIFICIAL
aggattcccaagctgtctcgtgtt

SEQ ID NO: 51 - DNA, 24 bp, ARTIFICIAL
  ARTIFICIAL
accatctgctgactgttcttgtgg

SEQ ID NO: 52 - DNA, 24 bp, ARTIFICIAL
  ARTIFICIAL
atgcagcgccaaacacatccattc

SEQ ID NO: 53 - DNA, 24 bp, ARTIFICIAL
  ARTIFICIAL
tttgagtttgacgcagcctaccac

SEQ ID NO: 54 - DNA, 21 bp, ARTIFICIAL
  ARTIFICIAL
gagaacaccacggcaatgttt

SEQ ID NO: 55 - DNA, 24 bp, ARTIFICIAL
  ARTIFICIAL
atgcaactctgacgttgatcccag

SEQ ID NO: 56 - DNA, 24 bp, ARTIFICIAL
  ARTIFICIAL
gactgcacatgtgttcttgagctg

SEQ ID NO: 57 - DNA, 24 bp, ARTIFICIAL
  ARTIFICIAL
agacaggttcttctggctgaaggt

SEQ ID NO: 58 - DNA, 24 bp, ARTIFICIAL
  ARTIFICIAL
ttcaatgccagatggcaaggttgg

SEQ ID NO: 59 - DNA, 22 bp, ARTIFICIAL
  ARTIFICIAL
tcccaaccatcattcaccctga

SEQ ID NO: 60 - DNA, 22 bp, ARTIFICIAL
  ARTIFICIAL
tgttgctgggagtgtacaggaa

SEQ ID NO: 61 - DNA, 24 bp, ARTIFICIAL
  ARTIFICIAL
tggaagctctaacactggagccaa

SEQ ID NO: 62 - DNA, 24 bp, ARTIFICIAL
  ARTIFICIAL
ctcctgatatccatccttgggtga

SEQ ID NO: 63 - DNA, 24 bp, ARTIFICIAL
  ARTIFICIAL
acttctcctgcaacaagttccctg

SEQ ID NO: 64 - DNA, 24 bp, ARTIFICIAL
  ARTIFICIAL
tgttgtccaagtttgtacgggagg